(19) United States
(12) Patent Application Publication
(10) Pub. No.: US 2018/0268940 A1
(43) Pub. Date: Sep. 20, 2018

(54) COMPOSITIONS AND METHODS FOR SPECIFIC BIOLOGICAL COGNITIVE FUNCTIONS IN NEURODEGENERATIVE DISEASES

(71) Applicant: Buck Institute for Research on Aging, Novato, CA (US)

(72) Inventor: Dale E. Bredesen, Novato, CA (US)

(21) Appl. No.: 15/762,324

(22) PCT Filed: Sep. 23, 2016
(86) PCT No.: PCT/US2016/053404

§ 371 Date: Mar. 22, 2018

Related U.S. Application Data
(60) Provisional application No. 62/233,221, filed on Sep. 25, 2015.

(51) Int. Cl. G16H 50/30; G06F 19/26; A61B 5/00

(52) U.S. Cl. CPC G16H 50/30; A61B 5/4088; G06F 19/26

(57) ABSTRACT

The invention described herein relates to novel personalized methods for treating, reducing or reversing cognitive decline utilizing a number of cognitive biological functions (factors), including metabolic parameters.

### CROSS-REFERENCE TO RELATED PATENT APPLICATION

This application claims the priority benefit of U.S. Provisional Patent Application Ser. No. 62/233,221, filed Sep. 25, 2015, the content of which is incorporated herein by reference in its entirety.

### FIELD OF THE INVENTION

This invention relates to compositions, methods of and systems for treating, reducing or reversing cognitive decline using specific biological cognitive functions.

### BACKGROUND OF THE INVENTION

Dementia (including cognitive decline and memory loss) is one of the most significant global healthcare problems, with over 30 million symptomatic individuals, and many more likely to be in the decades-long pre-symptomatic phases (World Alzheimer Report, 2009, www.alz.co.uk/research/files/WorldAlzheimerReport.pdf). In the United States alone, over five million people suffer from Alzheimer's disease (AD), at an estimated annual cost of $200 billion, and a projection for 13 million patients by 2050. The high prevalence of AD is of particular concern because of the lack of success in developing effective therapeutics: in comparison to most classes of disease—from neoplasia to cardiovascular and cerebrovascular disease to osteoporosis to diabetes to mental illness—therapeutic development for AD has been, to date, a failure.

Cognitive decline is a major concern of the aging population, and Alzheimer's disease is the major cause of age-related cognitive decline, with approximately 5.4 million American patients and 30 million affected globally (Prince M A, Emiliano; Guerchet, Mablenn; Prina, Matthew, 2014; World Alzheimer Report 2014 United Kingdom: Alzheimer's Disease International). In the absence of effective prevention and treatment, the prospects for the future are of great concern, with 13 million Americans and 160 million globally projected for 2050, leading to potential bankruptcy of the Medicare system. Unlike several other chronic illnesses, Alzheimer's disease prevalence is on the rise, which makes the need to develop effective prevention and treatment increasingly pressing. Recent estimates suggest that AD has become the third leading cause of death in the United States (James B D, Leurgans S E, Hebert L E, Scherr P A, Yaffe K and Bennett D A. Contribution of Alzheimer disease to mortality in the United States. Neurology. 2014; 82:1045 1050), behind cardiovascular disease and cancer. Furthermore, it has been pointed out recently that women are at the epicenter of the Alzheimer's epidemic, with 65% of patients and 60% of caregivers being women (Shriver M. A Woman's Nation Takes on Alzheimer's. 2010; New York, USA: Alzheimer's Association). Indeed, a woman's chance of developing AD is now greater than her chance of developing breast cancer (2014 Alzheimer's Disease Facts and Figures. Special Report on Women and Alzheimer's Disease. USA: Alzheimer's Association, 2014; pp. 1 80).

There has been a number of failures in the development of neurodegenerative disease therapeutics. Patients with acute illnesses such as infectious diseases, or with other chronic illnesses, such as cardiovascular disease, osteoporosis, human immunodeficiency virus infection, and even cancer, have access to more effective therapeutic options than do patients with AD or other neurodegenerative diseases such as Lewy body dementia, frontotemporal lobar degeneration, and amyotrophic lateral sclerosis. In the case of Alzheimer's disease, there appears to be no single therapeutic that exerts anything beyond a marginal, unsustained symptomatic effect, with little or no effect on disease progression. Furthermore, in the past decade alone, hundreds of clinical trials have been conducted for AD, at an aggregate cost of billions of dollars, without success.

Therefore, there is a great need for effective therapeutic methodology, compositions and systems for addressing cognitive decline, including treatment, diagnosis and identification of factors that can be used to improve cognitive health. The invention described herein addresses these problems and provides additional benefits as well.

### SUMMARY OF THE INVENTION

The invention described herein addresses the problem of cognitive decline and the failure to date of any viable, sustainable therapeutic(s) for addressing and/or treating cognitive decline.

Accordingly, provided herein are methods for treating, reducing or reversing cognitive decline in an individual. The methods include steps of (a) assessing at least 6 factors of Table 1; and (b) optimizing these factors if they are abnormal, where the optimizing is achieved by performing one or more optimization approaches associated with the factors.

Also provided herein are methods for treating, reducing or reversing cognitive decline in an individual. The methods include steps of (a) assessing at least 6 factors of Table 1; and (b) optimizing these factors if they are abnormal, where the optimizing is achieved by performing one or more optimization approaches associated with the factors, thereby treating, reducing or reversing cognitive decline in the individual.

Also provided herein are methods for treating, reducing or reversing cognitive decline in an individual. The methods include steps of (a) assessing at least 6 factors of Table 1; and (b) optimizing these factors if they are abnormal, where the optimizing is achieved by performing one or more optimization approaches associated with the factors, and where the factors include insulin resistance level, inflammation level, hormone status, homocysteine level, cytoprotection level or any combination thereof.

Also provided herein are methods for treating, reducing or reversing cognitive decline in an individual. The methods include steps of (a) assessing at least 6 factors of Table 1; and (b) optimizing these factors if they are abnormal, where the optimizing is achieved by performing one or more optimization approaches associated with the factors, and where the factors include insulin resistance level, inflammation level, hormone status, methylation level, cytoprotection level or any combination thereof.

Also provided herein are methods for treating, reducing or reversing cognitive decline in an individual. The methods include steps of (a) assessing at least 6 factors of Table 1; and (b) optimizing these factors if they are abnormal, where the optimizing is achieved by performing one or more optimization approaches associated with the factors, and where the factors include insulin resistance level, inflammation level, hormone status, homocysteine level, methylation level, cytoprotection level or any combination thereof.

Also provided herein are methods of identifying additional treatment modalities in individuals in need thereof. The methods include steps of (a) assessing at least 6 factors of Table 1; (b) identifying the factors that are abnormal; and (c) providing additional treatment modality to match the factor(s) of step (b).

Further provided herein are methods for metabolic enhancement of neurodegeneration in an individual that include the steps of (a) assessing at least 6 factors of Table 1; (b) optimizing these factors if they are abnormal, where the optimizing is achieved by performing one or more optimization approaches associated with the factors.

In some embodiments, at least 7 factors are assessed in the methods described herein. In some embodiments, at least 8 factors are assessed in the methods described herein. In some embodiments, at least 9 factors are assessed in the methods described herein. In some embodiments, at least 10 factors are assessed in the methods described herein.

In some embodiments, factors assessed in the methods include insulin resistance level, inflammation level, hormone status, homocysteine level, methylation level, cytoprotection level or any combination thereof.

In some embodiments, the factors assessed in the methods include hs-CRP level, homocysteine level, vitamin D level, hormone status, albumin:globulin ratio, serum albumin level, glucose status, metal status, alcohol use, history of head trauma, history of drug use, current use of neuroactive medications, ApoE4 status or any combination thereof.

In some embodiments, the insulin resistance factor can include glucose status, fasting blood sugar level or any combination thereof.

In some embodiments, the inflammation level can include cs-CRP level, arachidonic acid (AA)/eicosapentaenoic acid (EPA) ratio, meningitis, or any combination thereof. In embodiments, the inflammation level can include cs-CRP level, arachidonic acid (AA)/eicosapentaenoic acid (EPA) ratio or combination thereof.

In some embodiments, the hormone status can include vitamin D3 level, estradiol level, progesterone level, testosterone level, free T3 level, free T4 level, reverse T3 level, TSH level, pregnenolone level, DHEA level, morning cortisol level or any combination thereof.

In some embodiments, the cytoprotection level can include heavy metal toxicity, mitochondrial function, methylation status or any combination thereof.

In some embodiments, the glucose status can include fasting glucose level, fasting insulin level, hemoglobin A1c level or any combination thereof.

In some embodiments, the metal status can include Cu:Zn ratio, RBC Mg level, serum Zn level, RBC Zn level, serum Cu level, heavy metal toxicity or any combination thereof.

In some embodiments, the individual has memory loss.

In some embodiments, the individual has a family history of neurodegenerative disease.

In some embodiments, the individual has a neurodegenerative disease.

In some embodiments, the individual has Alzheimer's disease.

In some embodiments, the individual's health has not been improved on a monotherapy treatment plan. In some embodiments, the monotherapy treatment plan can include donepezil, memantine, rivastigmine, galantamine, huperzine A, a BACE inhibitor, an anti-amyloid antibody or any combination thereof.

Also provided herein are computer-implemented methods for implementation by one or more data processors forming part of at least one computing device to facilitate treating, reducing or reversing cognitive decline. The methods include receiving, at the one or more data processors, patient parameters of a patient, the patient parameters associated a set of physiological characteristics of the patient; comparing, at the one or more data processors, the patient parameters with predefined ranges for the set of physiological characteristics; and determining, at the one or more data processors, a memory loss risk factor for the patient based on the comparison.

In some embodiments, the set of physiological characteristics of the patient includes at least 6 factors of Table 1.

In some embodiments, the methods further include determining, by the one or more data processors, a memory loss treatment plan based on the patient parameters that exceed the predefined ranges for the associated physiological characteristics. For example, determining a memory loss treatment plan includes optimizing the set of physiological characteristics if the patient parameters are abnormal, where the optimization is achieved by performing one or more optimization approaches associated with the set of physiological characteristics.

In some embodiments, the methods further include presenting, through a graphical user interface, the memory loss treatment plan.

In some embodiments, determining the memory loss risk factor further includes determining, for individual ones of the patient parameters an amount that the individual patient parameter exceeds the predefined range for the associated physiological characteristic.

In some embodiments, determining the memory loss risk factor further includes aggregating the patient parameters that exceed the predefined ranges for the associated physiological characteristics.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference in their entirety for all purposes. The references cited herein are not admitted to be prior art to the claimed invention. In the case of conflict, the present specification, including definitions, will control. In addition, the materials, methods and examples are illustrative only and are not intended to be limiting.

Other features and advantages of the invention will be apparent from the following detailed description and claims.

### SUMMARY OF THE DRAWINGS

The accompanying drawings, which are incorporated in and constitute a part of this specification, show certain aspects of the subject matter disclosed herein and, together with the description, help explain some of the principles associated with the current description. In the drawings,

FIG. 1A-1D are illustrations of graphical user interfaces supporting features consistent with the present description; and,

FIG. 2 is a diagram illustrating aspects of a system showing features consistent with implementations of the present description.

### DETAILED DESCRIPTION

The invention described herein addresses the problem of cognitive decline and the failure to date of any viable, sustainable therapeutic(s) for addressing and/or treating cognitive decline.

Accordingly, provided herein are methods for treating, reducing or reversing cognitive decline based on a multiple-component therapeutic system. The multiple-component therapeutic methods described herein effectively utilize a number of cognitive biological functions (factors), including metabolic parameters. Without being bound by theory, it is believed that the overall balance underlies the disease pathogenesis, and as such, the more factors that are assessed, the more powerful the methods will be. In other words, the methods described herein utilize a network-based therapeutics approach, rather than a single target-based approach. As shown in the Example 1, the therapeutic methods and systems have demonstrated superior outcome of this approach on reversing the cognitive decline in patients.

Unlike previous known therapeutics, the therapeutic methods described herein aim to optimize metabolic parameters (factors), rather than to normalize them. The methods also address as many of the network components as possible-components/factors involving in pathogenesis network of cognitive decline or memory loss. Because the underlying network features a threshold effect, once enough of the network components have been impacted, the pathogenetic process of the disease would be halted or reversed. Therefore, even though it is not expected that most patients will be able to follow every single step of the treatment protocol (i.e., to optimize all the factors of the therapeutic methods described herein), as long as enough steps are followed to exceed the threshold, that should be sufficient. In addition, the methods are personalized, based on the contributory laboratory values affecting the plasticity network; and is computationally intensive, since many physiological data points are analyzed, interdependent network-component status is assessed, and many interventions are prioritized to determine the therapeutic program. The methods also use iterative program, so that there is continued optimization over time. For each network component/factor utilized in the methods, the goal is to address it in a way as physiological and as far upstream as possible.

In some embodiments, the methods described herein assess at least 1 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59) factors of Table 1 and Table 2. Specific methods for assessing these factors (i.e., performing an assay to determine the level/status/function of each factor) are described below.

In some embodiments, the methods described herein assess at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59) factors of Table 1 and Table 2.

In some embodiments, the methods described herein assess at least 10 (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59) factors of Table 1 and Table 2.

In some embodiments, the factors assessed in the methods described herein include insulin resistance level, inflammation level, hormone status, homocysteine level, methylation level, cytoprotection level or any combination thereof.

In some embodiments, the factors assessed in the methods described herein include insulin resistance level, inflammation level, hormone status, homocysteine level, cytoprotection level or any combination thereof.

In some embodiments, the factors assessed in the methods described herein include insulin resistance level, inflammation level, hormone status, methylation level, cytoprotection level or any combination thereof.

In some embodiments, the insulin resistance factor can include glucose status, fasting blood sugar level or any combination thereof.

In some embodiments, the inflammation level can include cs-CRP level, arachidonic acid (AA)/eicosapentaenoic acid (EPA) ratio, meningitis, or any combination thereof. In embodiments, the inflammation level can include cs-CRP level, arachidonic acid (AA)/eicosapentaenoic acid (EPA) ratio or combination thereof.

In some embodiments, the hormone status can include vitamin D3 level, estradiol level, progesterone level, testosterone level, free T3 level, free T4 level, reverse T3 level, TSH level, pregnenolone level, DHEA level, morning cortisol level or any combination thereof.

In some embodiments, the cytoprotection level can include heavy metal toxicity, mitochondrial function, methylation status or any combination thereof.

In some embodiments, the glucose status can encompass fasting glucose level, fasting insulin level, hemoglobin A1c level or any combination thereof. In some embodiments, the metal status can encompass Cu:Zn ratio, RBC Mg level, serum Zn level, RBC Zn level, serum Cu level, heavy metal toxicity or any combination thereof.

In some embodiments, the factors include hs-CRP level, homocysteine level, vitamin D level, hormone status, albumin:globulin ratio, serum albumin level, glucose status, metal status, alcohol use, history of head trauma, history of drug use, current use of neuroactive medications, ApoE4 status or any combination thereof.

Accordingly, in one embodiment, the methods for treating, reducing, or reversing cognitive decline described herein include: (a) assessing at least 6, at least 7, at least 8, at least 9, or at least 10 factors of Table 1 and Table 2; (b) optimizing these factors if these factors are abnormal, thereby treating, reducing, or reversing cognitive decline in an individual. The abnormal status of each factor is listed in Table 1. The optimizing for each factor is achieved by performing one or more optimization approaches associated with that factor. The method may further include identifying a subject having or at risk of developing a cognitive decline.

In another embodiment, the invention provides methods of identifying additional treatment modalities in an individual in need thereof. The methods include (a) assessing at least 6, at least 7, at least 8, at least 9, or at least 10 factors of Table 1 and Table 2; (b) identifying the factors that are abnormal; and (c) providing additional treatment modality to match the factor(s) of step (b). The method may further include identifying a subject having or at risk of developing a cognitive decline.

In another embodiment, the invention provides methods for metabolic enhancement of neurodegeneration in an individual. The methods include: (a) assessing at least 6, at least 7, at least 8, at least 9, or at least 10 factors of Table 1 and Table 2; (b) optimizing these factors if they are abnormal, where the optimizing for each factor is achieved by performing one or more optimization approaches associated with that factor. The method may further include identifying a subject having or at risk of developing neurodegeneration.

The step of assessing a factor of Table 1 and Table 2 in the methods described herein may include performing an assay to determine the level, status, and/or function of the factor of Table 1 and Table 2 and concluding if the level, status, and/or function of the tested factor is abnormal.

As a general non-limiting rule, the optimization is to place the factors at the midpoint of the range or better (better is lower for homocysteine, for example, and higher for vitamin D, for example—in each case, better is more toward the anti-AD part of the range), such as: homocysteine<7, vitamin D 50-100, Cu:Zn from 0.8 to 1.2, or goals set in Table 1 and Table 2. It is the optimization of each parameter/factor that changes the balance from synaptoclastic to synaptoblastic, and thus reverses the cognitive decline in an individual.

The following table provides an exemplary approach for each factor, how one of skill in the art would recognize that factor would be in the abnormal zone and how to optimize that factor to bring it to an end point or range that would be beneficial to the individual that has cognitive decline or is suspected of having cognitive decline.

All numerical designations (e.g., percentage, pH, dose, and concentration, including ranges) are approximations which are varied (+) or (−) by increments of 1.0 or 0.1, as appropriate. It is to be understood, although not always explicitly stated that all numerical designations are preceded by the term “about.” It also is to be understood, although not always explicitly stated, that the reagents described herein are merely exemplary and that equivalents of such are known in the art.

TABLE 1

| | | Optimization | Goal |
|---|---|---|---|
| Factors | Abnormal Threshold | Approach | (Optimized Target) |
| ApoE4 status | Heterozygote? Homozygote? | DESS (Diet exercise, | |
| | 3x risk for heterozygote; 10- | sleep, stress | |
| | 12x for homozygote | reduction); anti- | |
| | | inflammatory diet, | |
| | | supplements and | |
| | | herbs; heal leaky | |
| | | gut/blood-brain | |
| | | barrier | |
| Homocysteine | Homocysteine > about 7 | M-B12, M-folate, | < about 7 umol/l |
| level | umol/l | P5P; TMG | |
| Serum Vitamin | Vitamin B12 < about 500 | Methylcobalamin | about 500 pg/ml- |
| B12 level | pg/ml | 1 mg po qd | about 2000 pg/ml |
| | | po, by mouth | |
| | | qd, every day | |
| High sensitivity C- | CRP > about 1.0 mg/l | Anti-inflammatory | < about 1.0 mg/l |
| reactive protein | | diet; curcumin; | |
| (hsCRP) level | | DHA/EPA; optimize | |
| | | hygiene | |
| Albumin/globulin | A/G ratio < about 1.8 | optimize hygiene, | about 1.8-about 2.8 |
| ratio | Albumin < about 4.5 g/dl | anti-inflammatory | A/G ratio; |
| Serum albumin | | diet, etc. | Albumin: about 4.5- |
| level | | | about 5.4 g/dl |
| arachidonic acid | AA:EPA ratio > about 3 | | AA:EPA ratio < |
| (AA)/ | | | about 3 |
| eicosapentaenoic | | | |
| acid (EPA) ratio | | | |
| Glucose status: | HgbA1c > about 5.6% | Low glycemic diet; | HgbA1c < about |
| hemoglobin A1c | Fasting insulin > about 6 uIU | Paleolithic diet | 5.6% |
| level; | GTT insulin? | | Fasting insulin: |
| fasting insulin | | | about 4 uIU or less |
| level; | | | |
| glucose tolerance | | | |
| test (GTT) | | | |
| Type of diet | Simple carbohydrates (CHO) | Cut out simple CHO | remove simple |
| | in diet | diet; taking several | carbohydrates from |
| | | low glycemic, low | diet. |
| | | inflammatory, low | Eat anti- |
| | | grain diets; | inflammatory diet, |
| | | | high in good fats |
| | | | (omega-3, |
| | | | polyunsaturated, |
| | | | monounsaturated, |
| | | | some saturated, no |
| | | | trans fats), no |
| | | | farmed fish, beef |
| | | | only grass fed, |
| | | | chicken only |
| | | | pastured, eggs only |
| | | | pastured. |
| Glucose status: | FBS > about 90 mg/dl | Low glycemic diet | FBS: about 90 mg/dl |
| Fasting blood | | | or less |
| sugar (FBS) level | | | |
| Hormone status: | Thyroid: TSH > about 2.0 | Armour Thyroid or | TSH < about 2.0 |
| Thyroid/thyroid | | related T7 | mIU/l |
| stimulating | | | |
| hormone (TSH) | | | |
| ratio | | | |
| Hormone status: | fT3 < about 3.2 pg/ml | Armour Thyroid or | about 3.2-about 4.2 |
| Free | RT3 > about 20 ng/dl | related | pg/ml fT3 |
| triiodothyronine | | | |
| (fT3) level; | | | |
| Reverse T3 (RT3) | | | |
| level | | | |
| Hormone status: | fT3/RT3 ratio < about 20 | Armour Thyroid, | fT3:RT3 > about 20 |
| fT3/RT3 | ratio | reduce stress, check | |
| | | Fe, B6, B12, D | |
| Hormone status: | Free T4 < about 1.3 ng/dl | Armour Thyroid or | Free T4: about 1.3- |
| Free thyroxine | | related | about 1.8 ng/dl |
| (T4) level | | | |
| Sleep quality | Sleep apnea/hypopnea | CPAP or related Rx | No sleep apnea or |
| | | | treated successfully |
| Hormone status: | Low androgen: | T or activators | Total T > about 500 |
| Androgen level | Total T < about 500 ng/dl | (Ageless Male, etc.) | ng/dl |
| | Free T < about 6.5 ng/dl | | Free T > about 6.5 |
| | T, testosterone | | ng/dl |
| Hormone status: | Low estradiol or | Bioidentical estrogen | GOAL (for women): |
| Estradiol level | Post-menopausal: | trans-mucosal | E2: 80-150 pg/ml |
| | E2 < about 100 pg/ml | | estradiol (discuss |
| | E2:P > about 300 | | with physician, |
| | Hysterectomy at <41 year | | especially if over 10 |
| | old | | years past |
| | E2, estradiol; | | menopause); |
| | P, progesterone | | Progesterone: about |
| | | | 1-about 10 pg/ml; |
| | | | use bioidentical for |
| | | | both |
| Hormone status: | Low pregnenolone: < about | Pregnenolone | pregnenolone: about |
| Pregnenolone level | 20 ng/dl | | 50-about 150 ng/dl |
| Vitamin D level | Vitamin D < about 30 ng/ml | Vitamin D (100s | Vitamin D: about |
| | | rule) | 50-about 100 ng/ml |
| History of head | History of head trauma | Anti-tau | |
| trauma | LOC? | (nicotinamide? | |
| | LOC, loss of conscious | Lithium?); trophic | |
| | | environment | |
| Diabetes status | Diabetes | Low glycemic; | |
| | | metformin? | |
| Current use of | Taking neuroactive | DC neuroactive | |
| Neuroactive | medications | medications | |
| medications | Which? (Benzodiazepines? | | |
| | Statins? Antihypertensives? | | |
| | Antihistamines? Proton pump | | |
| | inhibitors? Antidepressants? | | |
| | Etc.) | | |
| History of drug use | History of drug use (Opiates? | Discontinue drug use | |
| | Cocaine? Etc.) | | |
| Metabolic health | Having metabolic syndrome: | DESS | |
| | Increased glucose, | | |
| | cholesterol, blood pressure | | |
| Cholesterol level | Cholesterol: | DESS | Cholesterol: |
| | > about 225 mg/dl | | about 170-about |
| | < about 150 mg/dl | | 225 mg/dl |
| HDL: total | Abnormal HDL: total | DESS | HDL: total |
| cholesterol ratio | cholesterol ratio > about 3.5 | | cholesterol ratio: |
| | | | about 3.5 or lower |
| Menopausal status | Post-menopausal | HRT or maca | |
| Andropausal status | Post-andropausal | T or related | |
| Diet | Taking high simple CHO diet | Discontinue simple | |
| | | CHO; induce | |
| | | autophagy (fasting) | |
| Metal status: | High free Cu: | Zn, B6, lipoic acid, | free copper < about |
| free copper (Cu) | (Cu-3x ceruloplasmin) > | ascorbate, reduce Cu, | 30 mcg/dl |
| | about 30 mcg/dl | Mn | (copper minus 3x |
| | | | ceruloplasmin) |
| Metal status: | Low Zn: | Zn 50 mg; ascorbate | Zinc: about 90- |
| Serum Zinc (Zn) | Zn < about 100 mcg/dl | 1 g | about 110 mcg/dl |
| level | Zn/free Cu < about 7 | | |
| Metal status: | Cu:Zn > about 1.3 | Zn 50 mg; ascorbate | Cu:Zn-between |
| Cu/Zn ratio | | lg | about 0.8 and about |
| | | | 1.2 |
| Metal status: | High Ca²⁺ | Mg; reduce Ca | Ca²⁺: about 9-about |
| Ca²⁺ level | Ca²⁺ > about 10.4 mg/dl | | 10 mg/dl |
| Metal status: | Low RBC Mg: | MgT 2 g po qhs | RBC Mg: about 5.2- |
| RBC Magnesium | RBC Mg < about 5.2 mg/dl | Po, by mouth | about 6.0 mg/dl |
| (Mg) level | | Qhs, one dose at | |
| | | bedtime | |
| Seed oil use | Using seed oil: | Discontinue seed | no seed oil use (no |
| | Coconut oil, olive oil, etc. | oils, use cold pressed, | oil with heat |
| | Check breath ethane | add vitamin E 400 | processing, such as |
| | | IU. | palm) |
| Vitamin E level | Vitamin E < about 10 mg/l | Vit E about 400- | Vitamin E: about |
| | | about 800 IU | 15-about 25 mg/l |
| Family history of | Family history of dementia: | Note in eval. | |
| dementia | Type? | | |
| | Age of onset? | | |
| Gene mutations | Mutation in APP, PS1, or | | |
| | PS2, PGRN, c9ORF, Tau | | |
| Dental (or other) | Poor dental (or other) | Get electric brush | No amalgams |
| hygiene | hygiene: | and flosser; peroxyl; | (removal by dentists |
| | presence of amalgams; | nails; cleaning | trained for safe |
| | periodontitis; | sinuses (sinus | amalgam removal); |
| | active caries | cleanses, such as | no periodontitis; use |
| | | Neti-pot or saline | of flossing, |
| | | spray or similar) | automatic |
| | | | toothbrush, and |
| | | | high-pressure water |
| | | | to optimize oral |
| | | | hygiene. |
| Thiamine or other | Thiamine or other vitamin | Replace | Thiamine about |
| vitamin level | deficiency | | 20 mg per day |
| Alcohol use | EtOH use: | Keep EtOH to 1 oz/d | no more than one |
| | Amount? | | glass of wine per |
| | Blackouts? | | day, or equivalent |
| | Seizure? | | |
| | Temporal association? | | |
| Vascular health | Having vascular disease | Ornish diet | |
| Toxin exposure | Having toxin exposure: DDE | Discontinue | Avoid of toxin |
| | levels high | exposure; getting | exposure; getting |
| | | specific treatment | treatment |
| Metal status: | Pb, Hg, Cd, Fe toxicity | Chelate, Rx | No evidence of |
| Heavy metal | Pb, lead | | heavy metal toxicity |
| toxicity | Hg, mercury | | (if sensitive testing |
| | Cd, cadmium | | by Quicksilver, less |
| | Fe, iron | | than 25% ile for all |
| | | | toxic metals) |
| Mitochondrial | Mitochondrial damage | | Avoidance of |
| function | (antibiotics, statins, | | mitochondrial toxins |
| | griseofulvin, AZT, | | |
| | acetaminophen, NSAIDS, | | |
| | cocaine, methamphet, L- | | |
| | DOPA, EtOH, ApoE4) | | |
| Kidney function | Renal insufficiency: | Evaluate re etiology | Creatinine < about |
| | Creatinine > about 1.5 mg/dl | Getting treatment for | 1.5 mg/dl |
| | | renal insufficiency | |
| Liver function | Hepatic insufficiency: | Evaluate re etiology | |
| | Serum albumin < about | Getting treatment for | |
| | 4.3 g/dl, | hepatic insufficiency | |
| | sometimes high bilirubin, | | |
| | sometimes high liver | | |
| | function tests, | | |
| | sometimes prolonged | | |
| | prothrombin time or partial | | |
| | thromboplastin time. | | |
| Hypoxia or | Having hypoxia or | Correct ABG | |
| hypercarbia or | hypercarbia or COPD | | |
| COPD | | | |
| Stress level | Stressed: | Stress reduction; | about 10-about 15 |
| | AM Cortisol > about 15 | rhodiola, etc. | mcg/dl for AM |
| | mcg/dl | | cortisol |
| BMI | BMI > about 25 | DESS | BMI: about 18- |
| | | | about 24 |
| Sleep hours | Sleep < about 7 hr/night | Increase; melatonin | Sleep about 8 hours |
| | | plus 5HTP if | per night; take |
| | | ruminating | melatonin 0.5 mg |
| | | If need sleeping pill, | each night; if |
| | | may occasionally use | awakening and |
| | | trazodone (SARI | ruminating, take |
| | | with sleep effects and | tryptophan 500 mg at |
| | | anxiolytic), Belsomra | night (unless on |
| | | (10 mg; but may have | anti-depressant, in |
| | | amnestic effects and | which case avoid |
| | | half-life 12 hrs), | tryptophan) |
| | | Xanax (short term, if | |
| | | awaken), Benadryl, | |
| | | Restoril | |
| Methylation status | Methylation defects on | Methyl-B12, methyl- | |
| | MTHFR gene (e.g., C677T) | folate; methylation | |
| | | treatment | |
| Herpes simplex 1 | Seropositive for Herpes | Acyclovir | |
| | simplex 1 | | |
| Headaches | Having headaches | Imaging, CSF | |
| | | (cerebrospinal fluid | |
| | | for detecting AD | |
| | | biomarkers and | |
| | | meningitis, | |
| | | encephalitis, other | |
| | | inflammatory CNS | |
| | | conditions) | |
| Mycotoxin | Mycotoxin exposure: | Water leak; diagnosis | No evidence of mold |
| exposure | Stachybotrys (“toxic black | of the source | exposure (ERMI |
| | mold”) | | score < about 2, |
| | | | serum C4a < about |
| | | | 2800) |
| Meningitis | Having meningitis | Treatment | |
| History of cancer | History of cancer | metastases to brain | |
| | (possibility of metastases to | | |
| | brain causing cognitive | | |
| | decline) | | |
| Gluten sensitivity | Gluten intolerance | Strict avoidance of | Negative Cyrex |
| | Check Cyrex Arrays 3, 4. | gluten +/− other | Array 2, 3, 20 |
| | Gut leak assay. | grains. | |
| GI health | Intestinal permeability | Probiotics, prebiotics, | |
| | | L-glutamine or | |
| | | colostrum/PRP | |

TABLE 2

| Goal | Approach | Selection | Comments |
|---|---|---|---|
| FOUNDATION | | | |
| Diet | Examples: | | Minimize glycemic |
| | “Eat to Live” or | | index (no sugar or bread |
| | “Blood Sugar Solution” or | | or simple |
| | “Grain Brain.” | | carbohydrates); no food |
| | Include nuts, blueberries, | | for 12 hours at night, or |
| | grapes, fish (animal | | 3 hrs before bed; add |
| | protein as condiment), | | coconut oil 1 teaspoon 1- |
| | minimize grains, test for | | 3x/day. Probiotics; beef |
| | gluten sensitivity (Cyrex | | should be grass-fed; fish |
| | Array 3). | | not farmed. |
| Reduce insulin | D-ribose, cinnamon ¼ tsp, | | |
| resistance | chromium picolinate, | | |
| | berberine (DPP4 inhibitor, | | |
| | 300-600 mg tid with meals | | |
| | to coincide with glu | | |
| | spike), Victoza-see list | | |
| | after table; biotin 2 mg tid; | | |
| Exercise | 3-6x/wk, 30′; get heart | | |
| | rate up, but be careful, | | |
| | increase slowly. | | |
| Stress Reduction | Personalized-yoga or | | |
| | meditation or music, etc. | | |
| Sleep | 8 hr sleep per night; | | |
| | melatonin 0.5 mg po qhs; | | |
| | Trp 500 mg po 3x/wk if | | |
| | awakening. Exclude sleep | | |
| | apnea. | | |
| Autophagy | Fast 12 hr each night, | | |
| | including 3 hr prior to | | |
| | bedtime. | | |
| Brain Stimulation | Posit or related | | |
| Hormone Balance | Optimize fT3, fT4, E2, T, | | |
| | progesterone, | | |
| | pregnenolone, cortisol | | |
| GI health | Probiotics; L-glutamine if | | |
| | leaky gut | | |
| SPECIFIC TX/RX | | | |
| [symbol] Aβ production (net) | Galangin, Bacopa | | |
| | monniera, F03, berberine, | | |
| | hydantoin BACE | | |
| | inhibitors (FAH), ?J03, | | |
| | ?clonidine, rapamycin, | | |
| | fasting | | |
| [symbol] Aβ oligomer- | Inositol, curcumin, | | |
| ization | Prosetta compounds | | |
| [symbol] BDNF | Exercise (aerobic), Posit | | |
| | (or Lumosity) | | |
| [symbol] NGF | Hericium erinaceus, | | |
| | ALCAR | | |
| [symbol] G-CSF | Coffee 5 cups? | | |
| [symbol] cAMP | 5HT1AR agonists (use | | |
| | Trp?); xanthines; | | |
| | hormones | | |
| [symbol] ADNP | (Davunetide) | | |
| Activate PPAR γ | Quercetin | | |
| [symbol] p-tau | Nicotinamide, Li, C31 [symbol] | | |
| | (F03, J17) | | |
| [symbol] homocysteine | B6, B12, folate, TMG | | |
| Build synapses | Citicoline, DHA, EPA, P- | | |
| | Ser | | |
| [symbol] 4/2 | F03, ASBI, FAH; IN | | |
| | disulfiram? | | |
| [symbol] Aβ breakdown | [symbol] insulin; [symbol] neprilysin | | |
| | (E2; other?) | | |
| [symbol] A/G ratio | “Super Hygene,” diet (low | | |
| | CHO, low sat'd fat), anti- | | |
| | inflam. | | |
| [symbol] inflammation | Hygiene, curcumin, herbs, | | |
| | ω3, diet, ginkgo, | | |
| | ashwagandha? Restrict | | |
| | food if sensitivities | | |
| | identified. | | |
| [symbol] GSH | NAC | | |
| [symbol] antioxidants | Se, Vit E, blueberry, | | |
| | astaxanthin, NAC | | |
| [symbol] Fe, Cu; [symbol] Zn | Check Fe, Cu, Zn; | | |
| | normalize | | |
| [symbol] CBF | Ginkgo, Fo-ti | | |
| [symbol] ACh | Huperzine A, Fo-ti, | | |
| | ?Bacopa? Choline, P- | | |
| | Choline, Rhodiola | | |
| [symbol] α7 signaling | F03 | | |
| [symbol] Aβ transport | Ashwagandha | | |
| [symbol] Aβ clearance | [symbol] albumin? Ashwagandha | | |
| | via receptors | | |
| [symbol] ApoE4 effect | F03, ?disulfiram-like? | | |
| [symbol] GABA? | F04? | | |
| [symbol] NMDA? | Huperzine A? F04? | | |
| | Memantine | | |
| Normalize hormones | (E2, prog., T, T7, | Include iodine, especially | |
| | pregnen., reduce cortisol) | if from iodine-poor region | |
| | | such as Great Lakes. | |
| [symbol] vitamin D | Vitamin D3 | | |
| [symbol] pro-NGF | ?add LM31 re p75 | | |
| | binding? | | |
| [symbol] caspase-6 | (?IN peptide?) | | |
| [symbol] N-APP | ASBI; trophic balance | | |
| [symbol] memory | B1, aniracetam, | | |
| | vinpocetine, F03, Mg, | | |
| | citicoline+ omega 3 | | |
| [symbol] Energy | Coconut oil, ALCAR, | | |
| | creatine | | |
| [symbol] Mitochondrial | α-lipoic acid, ALCAR, | | Avoid Mt toxins: |
| function | NAC, nicotinamide | | antibiotics, statins (these |
| | riboside 100 mg, Mg, Mn, | | reduce CoQ), |
| | B1, B2, B3, ubiquinol or | | griseofulvin, AZT, |
| | CoQ10 (LEF has with | | acetaminophen, |
| | shilajit for absorption; also | | NSAIDS, cocaine, |
| | ubiquinol, not | | methamphet, L-DOPA, |
| | ubiquinone). | | EtOH |
| [symbol] Mitochondrial | CoQ (300 mg), α-lipoic | | |
| protection | acid, Mt-targeted NAC | | |
| [symbol] Mitochondrial | PQQ (pyrroloquinoline | | PQQ is Aox, combines |
| biogenesis | quinone) 10-20 mg/d | | with CoQ for protection, |
| | | | inhibits amyloid fibrils, |
| | | | as well as Mt biogen. |
| [symbol] attention, focus | F03, pantothenate | | |
| Adaptogen | Ashwagandha | | |
| [symbol] SirT1/SirT2 | Resveratrol (clonidine? | | |
| | A03?) | | |
| [symbol] autophagy and | Fasting qhs; rapamycin- | | |
| mitophagy | like? Trehalose additive | | |
| | with rapa or low-dose | | |
| | (25 mg) resveratrol; | | |
| | Honokiol or magnolia | | |
| | extract; resveratrol low | | |
| | dose (25 mg) for Abeta | | |
| | phagocytosis | | |
| [symbol] AGE | Benfotiamine, alpha-lipoic | | |
| | acid, cinnamon | | |
| [symbol] Telomere length? | TA-65? | | |
| [symbol] Repair? | hGH? Secretagogues? (L- | | |
| | glutamine, Arg) | | |
| Bind D2 receptors | Bromocriptine or | | |
| | quinpirole (D2-3 specific); | | |
| | or [symbol] DA with Deprenyl | | |
| Bind D1 receptors to | ARR compound, D1 | | |
| increase ACh release | binding | | |
| [symbol] Cortical | DHA, vit. D, normalize | | |
| myelination | dopamine, normalize ACh | | |
| [symbol] glial scarring | Chondroitinase ABC | | |

In some embodiments, one of the factors of the methods may include ApoE4 status. The gene, APOE, is mapped to chromosome 19 in a cluster with Apolipoprotein C1 and the Apolipoprotein C2. The APOE gene consists of four exons and three introns, totaling 3597 base pairs. APOE is 299 amino acids long and contains multiple amphipathic α-helices. APOE is polymorphic, with three major alleles: ApoE2 (cys112, cys158), ApoE3 (cys112, arg158), and ApoE4 (arg112, arg158). Genomics of ApoE4 can be determined according to any known method in the art. Individuals with heterozygous ApoE4 allele have three-time higher risk for cognitive decline and individuals with homozygous ApoE4 allele have 10-to-12-time higher risk for cognitive decline than the individuals without ApoE4 allele. Diet, exercise, sleep and stress reduction are key to optimize this factor. Alternatively, this factor can be optimized by taking anti-inflammatory diets, supplements and herbs.

In some embodiments, one of the factors of the methods may include homocysteine level. Homocysteine level can be determined via any methods known in the art, which measures the amount of the amino acid homocysteine in the blood. Most laboratories report normal homocysteine levels in the blood between 4 and 15 micromoles/liter (μmol/L). In the claimed methods, however, when the homocysteine level is greater than about 7 μmol/l (e.g., about 7, 8, 9, 10, 11, 12, 13 μmol/l or higher) in the blood, an optimization is required for this factor. Such optimization can be achieved by, but is not limited to, taking M-B12, M-folate, pyridoxal-5-phosphate (P5P), and/or trimethylglycine (TMG), until the homocysteine level is less than about 7 μmol/l (e.g., about 7, 6, 5, 4, 3, 2, 1 μmol/l or less) in the blood.

In some embodiments, one of the factors of the methods may include serum vitamin B12 level. Serum vitamin B12 level can be determined by any methods known in the art, which measures the amount of vitamin B12 in the blood. Normally, values of less than 200 pg/mL are a sign of a vitamin B12 deficiency. Older adults with vitamin B12 levels between 200 and 500 pg/mL may also have symptoms of vitamin B12 deficiency. However, in the claimed methods, when the vitamin B12 level is lower than about 500 pg/ml (e.g., about 450, 400, 350, 300, 250, 200 pg/ml or less) in the blood, an optimization is required for this factor. Such optimization can be achieved by, but is not limited to, taking M-B12 (1 mg po qd), until the serum vitamin B12 level reaches about 500-about 2000 pg/ml (e.g., about 500, 600, 700, 800, 900, 1000, 1100, 1200, 1300, 1400, 1500, 1600, 1700, 1800, 1900, 2000 pg/ml) in the blood.

In some embodiments, one of the factors of the methods may include high sensitivity C-reactive protein (hsCRP) level. The CRP test is used by a health practitioner to detect inflammation. Any known method available in the art can be used to carry out the CRP test. When the hsCRP level in the blood is greater than about 1.0 mg/l (e.g., about 1.0, 1.1, 1.2, 1.3, 1.4, 1.5, 2.0, 2.5, 3.0, 4.0, 5.0, 10.0 mg/l or higher), an optimization is needed for this factor. Such optimization may be achieved by, but is not limited to, taking anti-inflammatory diet, taking curcumin, taking DHA, optimizing hygiene, until the hsCRP level is less than about 1.0 mg/l (e.g., about 1.0, 0.9, 0.8, 0.7, 0.6, 0.5, 0.4, 0.3, 0.2, 0.1 mg/l or lower).

In some embodiments, one of the factors of the methods may include albumin/globulin ratio and/or albumin level. Albumin and globulin level in the blood are frequently assessed as a part of an evaluation of a person's overall health status. Any known methods available in the art can be used to measure albumin and globulin level. The normal range for albumin is 3.5 to 5.5 g/dL or 35-55 g/liter. The normal range for serum globulin is usually 2.0 to 3.5 g/dL (grams per deciliter). In the claimed methods, however, when albumin is less than about 4.5 g/dl (e.g., about 4.5, 4.4, 0.4.3, 4.2, 4.1, 4.0, 3.5, 3.0, 2.5, 2.0 g/dl or less) in the blood and/or the albumin/globulin ratio is less than about 1.8, an optimization is needed for this factor. Such optimization may be achieved by, but is not limited to, optimizing hygiene, taking anti-inflammatory diet, until the ratio is between about 1.8 and about 2.8; and/or the albumin level reaches about 4.5-about 5.4 g/dl (e.g., about 4.5, 4.6, 4.7, 0.4.8, 0.4.9, 5.0, 5.1, 5.2, 5.3, 5.4 g/dl).

In some embodiments, one of the factors of the methods may include arachidonic acid (AA)/eicosapentaenoic acid (EPA) ratio, the ratio of two essential fatty acids in the blood. The AA/EPA ratio is an indication of the levels of cellular inflammation in the body. Any known methods available in the art can be used to measure these fatty acids in the blood. A ration of 0.2 or less is considered a normal AA/EPA reference ratio. However, in the claimed methods, when AA/EPA ratio is greater than about 3 (e.g., about 3, 3.5, 4, 4.5, 5, 5.5, 6, 6.5, 7, 8, 9, or higher), an optimization is needed for this factor. Such optimization can be achieved by, but is not limited to, increasing the intake of high-purity omega-3 fatty acid concentrates rich in EPA, taking anti-inflammatory diet until the AA/EPA ratio is less than about 3 (e.g., about 3, 2.5, 2, 1.5, 1 or lower).

In some embodiments, one of the factors of the methods may include glucose status. Glucose status can be determined by measuring hemoglobin A1c level, fasting insulin level, fasting glucose level in the blood, and insulin level in response to glucose tolerance test (GTT). Normally a hemoglobin A1c value is between 4% and 5.6% for people without diabetes and a fasting insulin level should be less than 25 mIU/L. In the claimed methods, when HgbA1c is greater than about 5.6% (e.g., about 5.7%, 5.8%, 5.9%, 6.0%, 6.50%, 7%, 7.5%, 8%, or higher) and/or the fasting insulin is higher than about 6 uIU (e.g., about 6, 7, 8, 9, 10, 11, 12, 13, 14, 15, 20, 25 uIU or higher) in the blood, an optimization is required for these factors. Such optimization can be achieved by, but is not limited to, taking low glycemic diet and/or taking Paleolithic diet, until HgbA1c is less than about 5.6% (e.g., about 5.6%, 5.4%, 5.3%, 5.2%, 5.1%, 5.0%, 4.9%, 4.8%, 4.7%, 4.6%, 4.5%, 4.0%, 3.5%, 3.0% or lower) and/or the fasting insulin is about 4 uIU or less (e.g., about 4, 3.5, 3, 2.5, 2, 1.5 uIU or less).

In some embodiments, one of the factors of the methods may include type of diet. When simple CHO in diet is taken, an optimization is needed for this factor. Such optimization may be achieved by cutting out simple CHO diet, taking several low glycemic, low inflammatory, low grain diets. The goal is to remove simple carbohydrates from diet, instead eating anti-inflammatory diet, high in good fats (omega-3, polyunsaturated, monounsaturated, some saturated, no trans fats), no farmed fish, beef only grass fed, chicken only pastured, eggs only pastured.

In some embodiments, one of the factors of the methods may include fasting blood sugar (FBS) level. FBS measures blood glucose after being fast for at least 8 hours. Normally, a fasting blood sugar level of less than 100 mg/dL (5.6 mmol/L) is considered normal. In the claimed methods, when FBS is greater than about 90 mg/dl (e.g., about 90, 100, 110, 120, 130, 140, 150, 200, 300 mg/dl or higher), an optimization is needed. Such optimization can be achieved by, but is not limited to, taking low glycemic diet, until FBS reaches about 90 mg/dl or less (e.g., about 90, 80, 70, 60, 50, 40, 30 mg/dl or less).

In some embodiments, one of the factors of the methods may include hormone status. Hormone status includes the status/level of vitamin D3, pregnenolone, free testosterone, free T3, reverse T3, free T4, TSH, progesterone, DHEA-S, IGF:IGF-BP3 ratio, fasting insulin, and/or morning (AM) cortisol. Any methods known in the art can be utilized to carry out the measurement of these hormones in blood or in saliva.

Vitamin D3, also known as cholecalciferol, is a secosteroid (i.e., a steroid molecule with one ring open). Cholecalciferol is inactive and it is converted to its active form by two hydroxylations in the liver first and later in the kidney. The active form then binds to vitamin D receptor, a nuclear receptor, that regulates the synthesis of hundreds of enzymes. Vitamin D3 level can be determined via any methods known in the art. A level between about 50-70 ng/ml is considered a normal vitamin D3 range. In the claimed methods, when vitamin D3 level is less than about 30 ng/ml, an optimization is needed. Taking vitamin D3 supplements or food rich of vitamin D3 can be one way to optimize the vitamin D3 level, until it reaches about 50 ng/ml to about 100 ng/ml (e.g., about 50, 55, 60, 65, 70, 75, 80, 85, 90, 95, 100 ng/ml).

Pregnenolone is a chemical substance that is a precursor to all steroid hormones. This test measures the amount of pregnenolone in the blood. When pregnenolone level is low (for example, lower than about 20 ng/dl (e.g., about 20, 19, 18, 17, 16, 15, 10, 5 ng/dl or lower)), an optimization is needed. Taking pregnenolone supplements can be one way to optimize the pregnenolone level, until it reaches about 50-about 150 ng/dl (e.g., about 50, 60, 70, 80, 90, 100, 110, 120, 130, 140, 150 ng/dl).

A testosterone test checks the level of this male hormone (androgen) in the blood. Total testosterone (“total T”) refers to all the testosterone in the body. Free testosterone (“Free T”) refers to the amount of testosterone that is bioactive, that is, ready for the body to use. When androgen level is low (for example, when total T is less than about 500 ng/dl (e.g., about 500, 450, 400, 350, 300, 250 ng/dl or lower) and/or when free T is lower than about 6.5 ng/dl (e.g., about 6.5, 6.4, 6.3, 6.2, 6.1, 6.0, 5.5, 5.0, 4.5 ng/dl or lower)), an optimization is needed. Taking testosterone supplements or activators (e.g., Ageless Male) may be used to optimize the androgen level, until total T reaches greater than about 500 ng/dl (e.g., about 500, 510, 520, 530, 540, 550, 600 ng/dl or higher) and/or free T reaches greater than about 6.5 ng/dl (e.g., about 6.5, 6.6, 6.7, 6.8, 6.9, 7.0, 7.5, 8 ng/dl or higher).

A TSH blood test is used to check for thyroid gland problems. TSH is produced when the hypothalamus releases a substance called thyrotropin-releasing hormone (TRH). TRH then triggers the pituitary gland to release TSH. Any methods known in the art can be utilized to measure thyroid and TSH level in the blood. When the thyroid/TSH ratio is greater than about 2 (e.g., about 2, 2.5, 3, 3.5, 4 or higher), an optimization is needed for this factor. Such an optimization may be achieved by, but is not limited to, taking Armour Thyroid or related T7 supplements until TSH reaches lower than about 2.0 mIU/l (e.g., about 2.0, 1.9, 1.8, 1.7, 1.6, 1.5, 1.4, 1.3, 1.2, 1.0 mIU/l or lower).

Triiodothyronine (T3) and T4 (thyroxine) are hormones produced by the thyroid gland. They help control the rate at which the body uses energy and are regulated by a feedback system. TSH stimulates the production and release of T4 (primarily) and T3. As needed, T4 is converted into T3 by the liver and other tissues. Most of the T4 and T3 circulates in the blood bound to protein, while a small percentage is free (not bound). There is another substance produced by the thyroid called RT3, which stands for Reverse T3, and it comes from the conversion of the storage hormone T4. Blood tests can measure total T4 (unbound plus bound), free T4, total T3 (bound plus unbound), or free T3, and RT3. When fT3 is lower than about 3.2 pg/ml (e.g., about 3.2, 3.1, 3.0, 2.9, 2.8, 2.7, 2.6, 2.5, 2.0 pg/ml or lower) and/or when RT3 is greater than about 20 pg/ml (e.g., about 20, 21, 22, 23, 24, 25, 30, 35, 40 pg/ml or higher) and/or when the ratio of fT3/RT3 is less than about 20 (e.g., about 20, 19, 18, 17, 16, 15, 10, 5 or lower) and/or when free T4 is greater than about 1.3 ng/dl (e.g., about 1.3, 1.4, 1.5, 1.6, 1.7, 1.8, 1.9, 2.0 ng/dl or higher), an optimization is needed for these factors. Such optimization may be achieved by taking Armour Thyroid or related hormone supplements, reducing stress, and/or checking iron, vitamin B6, vitamin B12 and vitamin D levels, until fT3 reaches about 3.2-about 4.2 pg/ml (e.g., about 3.2, 3.3, 3.4, 3.5, 3.6, 3.7, 3.8, 3.9, 4.0, 4.1, 4.2 pg/ml); and/or the ratio of fT3 to RT3 is greater than about 20 (e.g., about 20, 21, 22, 23, 24, 25, 30, 35 or higher); and/or free T4 reaches about 1.3-about 1.8 ng/dl (e.g., about 1.3, 1.4, 1.5, 1.6, 1.7, 1.8 ng/dl).

An estradiol test is a blood test that measures the amount of estradiol in the blood. It may also be called an E2 test. Estradiol is a form of the hormone estrogen. When the E2 level is lower than about 100 pg/ml (e.g., about 100, 95, 90, 85, 80, 75 pg/ml or lower), and/or the ratio of E2/progesterone is greater than about 300 (e.g., about 300, 310, 320, 330, 340, 350, 400, 450, 500 or higher), and/or the subject is post-menopausal; and/or the subject had hysterectomy at age younger than about 41 years old (e.g., about 41, 40, 39, 38, 37, 36, 35 or younger), an optimization is needed for estradiol. Such optimization can be achieved by taking bioidentical estrogen (trans-mucosal). Goal of optimization for women is: about 80-about 150 pg/ml of E2 (e.g., about 80, 90, 100, 110, 120, 130, 140, 150 pg/ml); and/or about 1-about 10 pg/ml of progesterone (e.g, about 1, 2, 3, 4, 5, 6, 7, 8, 9, 10 pg/ml).

Dehydroepiandrosterone sulfate (DHEAS) is a male sex hormone (androgen) that is present in both men and women. This test measures the level of DHEAS in the blood. When the DHEAS level is lower than about the 25^{th} percentile (e.g., about 25%, 24%, 23%, 22%, 21%, 20%, 15% or lower percentile) for gender, age, and test (saliva or serum, etc.), or higher than about the 90^{th} percentile (e.g., about 90, 91, 92, 93, 94, 95, 96, 97, 98, 99 percentile or higher), an optimization is needed. Such optimization may be achieved by taking DHEA (e.g., 25 mg by mouth each day, then re-check level in one month).

The ratio of the insulin-like growth factor (IGF) to IGF binding protein-3 (IGF-BP3) can also be one factor used for assessing hormone status of the method. When this ratio is in the lowest quartile, an optimization is needed. Such optimization may be achieved by the use of human growth hormone.

Morning cortisol level is another factor used for assessing hormone status of the methods. A cortisol level higher than about 15 mcg/dl (e.g., about 15, 16, 17, 18, 19, 20, 25, 30, 35 mcg/dl or higher) in the blood indicates that the subject is under stress and an optimization is required. Stress reduction may be achieved by personalized activities (e.g., yoga or meditation or music, etc.) and/or by taking supplements (such as Rhodiola). The goal for optimization is to reduce AM cortisol level to about 10-about 15 mcg/dl (e.g., about 10, 11, 12, 13, 14, 15 mcg/dl).

In some embodiments, one of the factors of the methods may include sleep quality. When the subject is suffering from sleep apnea/hypopnea, an optimization is needed. Treatment like continuous positive airway pressure (CPAP) or related prescription drugs may be used to optimize the sleep quality, until no sleep apnea or treated successfully.

In some embodiments, one of the factors of the methods may include vitamin D level. Vitamin D level can be determined according to any known methods available in the art. When vitamin D level is lower than about 30 ng/ml (e.g., about 30, 29, 28, 27, 26, 25, 24, 23, 22, 21, 20, 15, 10 ng/ml or lower), an optimization is needed. Taking vitamin D supplements is a preferred way to achieve such optimization until its level reaches about 50-about 100 ng/ml (e.g., about 50, 55, 60, 65, 70, 75, 80, 85, 90, 95, 100 ng/ml).

In some embodiments, one of the factors of the methods may include history of head trauma. When a subject has a history of head trauma and/or had loss of conscious due to head trauma, an optimization is needed. Such optimization can be achieved by taking anti-tau drugs (e.g., nicotinamide or lithium) or moving to trophic environment.

In some embodiments, one of the factors of the methods may include diabetes status. When a subject is suffering from diabetes, an optimization is needed. Taking low glycemic diet and/or taking metformin are exemplary approaches to achieve such optimization.

In some embodiments, one of the factors of the methods may include current use of neuroactive medications. When a subject is taking one or more neuroactive medications, an optimization is required. Exemplary neuroactive medications include benzodiazepines, statins, antihypertensives, antihistamines, proton pump inhibitors, antidepressants, etc. Discontinuing taking such medications is the preferred way to achieve such optimization.

In some embodiments, one of the factors of the methods may include history of drug use. When a subject has a history of drug use (such as opiates or cocaine), an optimization is required. Discontinuing such drug use is the preferred way to achieve such optimization.

In some embodiments, one of the factors of the methods may include metabolic health. When a subject had or is suffering from a metabolic syndrome, an optimization is required. Diet, exercise, sleep and stress reduction are the key to achieve such optimization.

In some embodiments, one of the factors of the methods may include cholesterol level. When cholesterol level is higher than about 225 mg/dl (e.g., about 225, 230, 235, 240, 245, 250, 300, 350, 400 mg/dl or higher) or lower than about 150 mg/dl (e.g., about 150, 140, 130, 120, 110, 100, 90, 80, 70, 60, 50 mg/dl or lower), an optimization is needed. Diet, exercise, sleep and stress reduction are the key to achieve such optimization until cholesterol level reaches about 170-about 225 mg/dl (e.g., about 170, 175, 180, 185, 190, 195, 200, 205, 210, 215, 220, 225 mg/dl).

In some embodiments, one of the factors of the methods may include HDL to total cholesterol ratio. An abnormal HDL/total cholesterol ratio of greater than 3.5 (e.g., about 3.5, 4, 4.5, 5, 5.5, 6, 6.5, 7, 8, 9, 10, 15 or higher) indicates that an optimization is required. Diet, exercise, sleep and stress reduction are the key to achieve such optimization until the ratio reduces to about 3.5 or lower (e.g., about 3.5, 3.4, 3.3, 3.2, 3.1, 3.0, 2.5, 2.0 or lower).

In some embodiments, one of the factors of the methods may include menopasusal status. When a subject is at the post-menopausal stage, an optimization by undertaking hormone replacement therapy (HRT) or taking maca is needed.

In some embodiments, one of the factors of the methods may include andropausal status. When a subject is at the post-andropausal stage, an optimization by taking testosterone or related supplements is needed.

In some embodiments, one of the factors of the methods may include metal status, such as Cu:Zn ratio, RBC Mg, serum zinc, RBC zinc, serum copper, heavy metal toxicity, iron. Methods for detecting metal concentration in the blood are well known in the art. An optimization of metal status is needed when a subject has one or more of the following measurement results: free Cu level is greater than about 30 mcg/dl (e.g., about 30, 31, 32, 33, 34, 35, 40, 45, 50 mcg/dl or higher); serum zinc level is lower than about 100 mcg/dl (e.g., about 100, 95, 90, 85, 80, 75, 70 mcg/dl or lower); the ratio Zn/free Cu is less than about 7 (about 7, 6, 5, 4, 3 or lower); the ratio Cu/Zn is greater than about 1.3 (e.g., about 1.3, 1.4, 1.5, 1.6, 1.7, 1.8, 1.9, 2.0 or higher); Ca²⁺ level is greater than about 10.4 mg/dl (e.g., about 10.4, 10.5, 10.6, 10.7, 10.8, 10.9, 11, 12, 13, 14, 15 mg/dl or higher); and RBC Mg level is lower than about 5.2 mg/dl (e.g., about 5.2, 5.1, 5.0, 4.9, 4.8, 4.7, 4.6, 4.5 mg/dl or lower). Increasing Zn, B6, lipoic acid, ascorbate, and/or Mg (such as Zn 50 mg or MgT 2 g per day) intake is preferred way to optimize the metal status. The goal is to optimize the metal status that is represented by the following numbers: free Cu less than about 30 mcg/dl (e.g., about 30, 29, 28, 27, 26, 25, 24, 23, 22, 21, 20, 15 mcg/dl or lower); serum Zn about 90-about 110 mcg/dl (e.g., 90, 91, 92, 93, 94, 95, 96, 97, 98. 99, 100, 101, 102, 103, 104, 105, 106, 107, 108, 109, 110 mcg/dl); Cu/Zn ratio about 0.8-about 1.2 (e.g., about 0.8, 0.9, 1.0, 1.1, 1.2); Ca²⁺ about 9-about 10 mg/dl (e.g., about 9.1, 9.2, 9.3, 9.4, 9.5, 9.6, 9.7, 9.8, 9.9, 10 mg/dl); RBC Mg about 5.2-about 6.0 mg/dl (e.g., about 5.2, 5.3, 5.4, 0.5.5, 5.6, 5.7, 5.8, 5.9, 6.0 mg/dl).

In addition, when a subject is suffering from heavy metal (Pb, Hg, Cd, Fe) toxicity, chelation therapy (such as DMPS, succimer, calcium-disodium EDTA) and relevant prescription drugs for treating heavy metal toxicity can be used to optimize this factor until no evidence of heavy metal toxicity.

In some embodiments, one of the factors of the methods may include seed oil use. When a subject is using seed oil, an optimization (which is to discontinue the use of seed oil and to use cold pressed oil; and to add vitamin E 400 IU) is needed, until no use of seed oil and no use of oil with heat processing (such as palm).

In some embodiments, one of the factors of the methods may include vitamin E level. When the vitamin E level is lower than about 10 mg/l (e.g., about 10, 9.5, 9.0, 8.5, 8, 7.5, 7.0, 6.5 mg/l or lower), an optimization is needed for the subject. Taking vitamin E 400-800 IU is a preferred way to achieve such optimization, which is when vitamin E reaches about 15-about 25 mg/l (e.g., about 15, 16, 17, 18, 19, 20, 21, 22, 23, 24, 25 mg/dl).

In some embodiments, one of the factors of the methods may include family history of dementia. Notes should be taken in the evaluation form if the subject has a family history of dementia. Specific questions such as type and age of onset should be further answered by the subject. Further evaluation of genetic background of the subject may be ordered by the health provider.

In some embodiments, one of the factors of the methods may include gene mutations, such as mutation in amyloid precursor protein (APP), Presenilin-1 (PS1), Presenilin-2 (PS2), Progranulin (PGRN), c9ORF (chromosome 9 open reading frame) and/or Tau. Existence of any such mutation indicates that the subject needs an optimization. These mutations can be detected according to any known methods in the art. Two groups of tests, molecular and cytogenetic, are used. In general, single base pair mutations are identified by direct sequencing, DNA hybridization and/or restriction enzyme digestion methods. Cytogenetics and molecular cytogenetics includes conventional karyotyping, fluorescence in situ hybridization (FISH), and comparative genomic hybridization (CGH). Molecular diagnostics provide a way for assessment of the genetic makeup of human; it combines laboratory medicine with molecular genetics to develop DNA/RNA-based analytical methods for monitoring human pathologies. A wide range of methods has been used for mutation detection, including (but is not limited to) polymerase chain reaction (PCR) and its versions, DNA microarray, DNA sequencing, Multiplex ligation-dependent probe amplification (MLPA), Single Strand Conformational Polymorphism (SSCP), Denaturing Gradient Gel Electrophoresis (DGGE), Heteroduplex analysis, and Restriction fragment length polymorphism (RFLP).

In some embodiments, one of the factors of the methods may include dental (or other) hygiene status. When a subject has a poor dental (or other) hygiene (such as presence of amalgam, suffering from periodontitis; or having active caries), an optimization is required. Such optimization may be achieved by getting electric tooth brush and flosser; using peroxyl mouth rinse; cutting nails regularly; and/or using sinus cleanses such as Neti-pot or saline spray or similar product). The goal is no amalgams (removal by dentists trained for safe amalgam removal); no periodontitis; use of flossing, automatic toothbrush, and high-pressure water to optimize oral hygiene.

In some embodiments, one of the factors of the methods may include thiamine or other vitamin level. If a subject has thiamine or other vitamin deficiency, an optimization is needed. Taking related vitamin supplements is a preferred way to achieve such optimization when consuming thiamine about 20 mg per day.

In some embodiments, one of the factors of the methods may include alcohol usage. Questions such as amount consumed per day, whether the patient had blackouts (and how often), whether the patient had seizure and whether the patient had temporal association with drinking can be asked to provide further insight of the patient about alcohol use. When a subject consumes more than about 1 oz (e.g., about 1, 2, 3, 4, 5, 6, 7, 8, 9, 10 oz or more) per day of alcohol, an optimization by reducing the consumption to less than 1 oz (e.g., about 1, 0.9, 0.8, 0.7, 0.6, 0.5 oz or less) per day is needed. The goal for this factor is no more than one glass of wine per day, or equivalent.

In some embodiments, one of the factors of the methods may include vascular health. When a subject has vascular disease, an optimization (such as taking Ornish diet) is needed. Ornish diet is a type of low fat diet available in the market (Carb: 65%, Protein: 15%, Fat: 20%). Vascular disease includes any condition that affects the circulatory system, e.g., peripheral artery disease, aneurysm, renal artery disease, Raynaud's phenomenon, Buerger's Disease, peripheral venous disease, varicose veins, etc.

In some embodiments, one of the factors of the methods may include toxin exposure. When a subject is exposed to high DDE, an optimization (such as discontinuation of the exposure or getting necessary treatment) is needed until completely avoidance of such toxin exposure and/or getting specific treatment. Without being bound by theory, it is believed that toxins are important contributors to Alzheimer's disease, which has not been addressed by the vast majority of doctors.

In some embodiments, one of the factors of the methods may include mitochondrial function. An optimization is required, when a subject is suffering from mitochondrial damage caused by taking antibiotics, statins, griseofulvin, azidothymidine (AZT), acetaminophen, NSAIDS, cocaine, methamphet, L-DOPA, EtOH, and/or ApoE4. Any methods known in the art can be used to detect mitochondrial damage, such as any methods for detecting reduced function of mitochondria or methods for analyzing mitochondrial DNA.

In some embodiments, one of the factors of the methods may include kidney function. Creatinine has been found to be a fairly reliable indicator of kidney function. Creatinine measurement is a routine test in the art. A creatinine level greater than about 1.5 mg/dl (e.g., about 1.5, 1.6, 1.7, 1.8, 1.9, 2.0, 2.5, 3.0, 3.5 mg/dl or higher) indicates renal insufficiency and requires an optimization for a subject. The optimization goal is to lower the creatinine level to about 1.5 mg/dl or less (e.g., about 1.5, 1.4, 1.3, 1.2, 1.1, 1.0, 0.9, 0.8, 0.7, 0.6, 0.5 mg/dl or less). Changing lifestyle (such as stop smoking, eating a healthy, low-fat, balanced diet), restricting salt intake, moderating alcohol intake, losing weight, doing exercise) and/or getting proper treatment are options to achieve this goal.

In some embodiments, one of the factors of the methods may include liver function. Serum albumin level less than about 4.3 g/dl (e.g., about 4.3, 4.2, 4.1, 4.0, 3.9, 3.8, 3.7, 3.6, 3.5 g/dl or less), sometimes high bilirubin, sometimes high liver function tests, sometimes prolonged prothrombin time or partial thromboplastin time may indicate hepatic insufficiency. Liver enzyme tests, formerly called liver function tests (LFTs), are a group of blood tests that detect inflammation and damage to the liver. They can also check how well the liver is working. Liver enzyme testing includes ALT, AST, alkaline phosphatase; true liver function tests (LFTs) include PT, INR, albumin, and bilirubin. Existence of hepatic insufficiency indicates that an optimization of this factor is needed for the subject. Changing lifestyle (such as stop smoking, eating a healthy, low-fat, balanced diet), moderating alcohol intake, doing exercise) and/or getting proper treatment are options to achieve this optimization.

In some embodiments, one of the factors of the methods may include diagnosis of hypoxia or hypercarbia or COPD. Having hypoxia or hypercarbia or COPD in a subject indicates that an optimization is needed for this factor. Correction of arterial blood gases (ABGs) is the preferred method to achieve such optimization. ABG is a collective term applied to three separate measurements—pH, Pco2, and Po2—generally made together to evaluate acid-base status, ventilation, and arterial oxygenation. Oxygen (O2) and carbon dioxide (CO2) are the most important respiratory gases.

In some embodiments, one of the factors of the methods may include stress level. As described above, morning cortisol level of greater than about 15 mcg/dl (e.g., about 15, 16, 17, 18, 19, 20, 25, 30, 35 mcg/dl or higher) indicates that a subject is under stress. Stress reduction may be achieved by personalized activities (e.g., yoga or meditation or music, etc.) and/or by taking supplements (such as Rhodiola). Optimization will be reached when the AM cortisol level is about 10-about 15 mcg/dl (e.g., about 10, 11, 12, 13, 14, 15 mcg/dl).

In some embodiments, one of the factors of the methods may include BMI. A BMI index that is greater than 25 (e.g., about 25, 26, 27, 28, 29, 30 or higher) suggests that an optimization is needed for this factor. Diet, exercise, sleep and stress reduction are the key to optimize this factor (i.e., to reduce the BMI to 18-24, e.g., about 18, 19, 20, 21, 22, 23, 24).

In some embodiments, one of the factors of the methods may include time of sleep. Less than about 7 hours of sleep per night indicates a required optimization. About 8-hour sleep per night or taking melatonin 0.5 mg po qhs; Trp 500 mg po 3×/week if awakening can be used to achieve this optimization.

In some embodiments, one of the factors of the methods may include status of methylene tetrahydrofolate reductase (MTHFR). Existence of methylation defects due to MTHFR C677T allele indicates a required optimization. To achieve the optimized methylation status, a subject can take Methyl-B12, methyl-folate or methylation treatment.

In some embodiments, one of the factors of the methods may include sensitivity of Herpes simplex 1. Seropositive for Herpes simplex 1 suggests a required optimization for this factor. Taking antiviral drugs that are effective for treating herpes (such as acyclovir, valaciclovir (valacyclovir), famciclovir, and penciclovir) is an option to optimize this factor (i.e., treating herpes).

In some embodiments, one of the factors of the methods may include headache. Having a headache is an indicator that an optimization is needed for this factor. Identifying the source that causes headache by imaging or detecting cerebrospinal fluid for AD biomarkers, other inflammatory CNS conditions and to exclude meningitis, encephalitis can be an approach to optimize this factor.

In some embodiments, one of the factors of the methods may include mycotoxin exposure. Having mycotoxin exposure indicates that an optimization is needed for this factor. Identifying (diagnosis) and eliminating the source for such exposure are approaches to optimize this factor, such us fixing the water leak. No evidence of mold exposure (ERMI score<about 2 (e.g., about 2, 1.9, 1.8, 1.7, 1.6, 1.5, 1.0 or less), serum C4a<about 2800 (e.g., about 2800, 2700, 2600, 2500, 400, 2300, 2200, 2100, 200, 1500, 100 or smaller)) is the optimization goal for this factor

In some embodiments, one of the factors of the methods may include meningitis. Meningitis is a disease caused by the inflammation of the protective membranes covering the brain and spinal cord known as the meninges. The inflammation is usually caused by an infection of the fluid surrounding the brain and spinal cord. Having meningitis is an indicator that an optimization is needed for this factor. Proper treatment for meningitis, which depends on the type of meningitis, is the preferred way to optimize this factor. For example, acute bacterial meningitis requires prompt treatment with intravenous antibiotics and, more recently, cortisone medications.

In some embodiments, one of the factors of the methods may include history of cancer. Having a history of cancer is an indicator that an optimization is needed for this factor. Metastases to brain may cause cognitive decline.

In some embodiments, one of the factors of the methods may include gluten sensitivity. Gluten sensitivity can be determined by Cyrex Array 3 or 4 and/or gut leak assay. A subject who is sensitive to gluten can optimize this factor by strictly avoiding any gluten-containing food.

In some embodiments, one of the factors of the methods may include GI health. Having intestinal permeability (which is the phenomenon of the gut wall in the gastrointestinal tract exhibiting permeability) suggests a required optimization of this factor. Taking probiotics, prebiotics, L-glutamine or colostrum/PRP is a preferred approach to optimize this factor. Colostrinin (also known as CLN, proline-rich polypeptides or PRP) is a naturally occurring mixture of proline-rich polypeptides derived from colostrum.

In some embodiments, one of the factors of the methods may include insulin resistance, inflammation level, hormone status, homocysteine level, methylation level, metal status, cytoprotection level, or any combination thereof.

In some embodiments, one of the factors of the methods may include hs-CRP level, homocysteine level, vitamin D level, hormone status, albumin:globulin ratio, serum albumin level, glucose status, metal status, alcohol use, history of head trauma, history of drug use, current use of neuroactive medications, ApoE4 status or any combination thereof.

In some embodiments, the insulin resistance factor can include glucose status, fasting blood sugar level or any combination thereof.

In some embodiments, the inflammation level can include cs-CRP level, arachidonic acid (AA)/eicosapentaenoic acid (EPA) ratio, meningitis, or any combination thereof. In embodiments, the inflammation level includes cs-CRP level, arachidonic acid (AA)/eicosapentaenoic acid (EPA) ratio or combination thereof.

In some embodiments, the hormone status can include vitamin D3 level, estradiol level, progesterone level, testosterone level, free T3 level, free T4 level, reverse T3 level, TSH level, pregnenolone level, DHEA level, morning cortisol level or any combination thereof.

In some embodiments, the cytoprotection level can include heavy metal toxicity, mitochondrial function, methylation status or any combination thereof.

In some embodiments, the glucose status can include fasting glucose level, fasting insulin level, hemoglobin A1c level or any combination thereof.

In some embodiments, the metal status can include Cu:Zn ratio, RBC Mg level, serum Zn level, RBC Zn level, serum Cu level, heavy metal toxicity or any combination thereof.

An anti-inflammatory diet refers to the Zone diet, which in general suggests: eating plenty of fruits and vegetables, minimizing saturated and trans fats, eating a good source of omega-3 fatty acids (such as fish or fish oil supplements, and walnuts), watching intake of refined carbohydrates (such as pasta and white rice), eating plenty of whole grains (such as brown rice and bulgur wheat), eating lean protein sources (such as chicken), cutting back on red meat and full-fat dairy foods; avoiding refined foods and processed foods; and spicing it up with ginger, curry, and other spices.

A low-glycemic diet is one that selects foods on the basis of minimal alteration of circulating glucose levels. Glycemic index (GI) and glycemic load (GL) are measures of the effect on blood glucose level after a food containing carbohydrates is consumed.

The Paleolithic diet is a diet based on the foods' ancient ancestors might likely have eaten, such as meat, nuts and berries, and excludes food to which they had not yet become familiar, like dairy.

The goal set in Table 1 and Table 2 for each factor is equivalent to the optimized target for each factor for the methods described herein. “ [symbol] ” in Table 2 indicates that the goal is to increase the level of that specific factor and “⬇” indicates that the goal is to decrease the level of that specific factor.

The term “subject” and “individual” are interchangeable here, and both refer to a mammal, including primates (e.g., human).

“Cognitive function” or “cognitive status” refers to any higher order intellectual brain process or brain state, respectively, involved in learning and/or memory including, but not limited to, attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, and expressing an interest in one's surroundings and self-care.

In humans, cognitive function may be measured, for example and without limitation, by the clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB) or the Sandoz Clinical Assessment-Geriatric (SCAG). See Folstein et al., J Psychiatric Res 12: 189-98, (1975); Robbins et al., Dementia 5: 266-81, (1994); Rey, L'examen clinique en psychologie, (1964); Kluger et al., J Geriatr Psychiatry Neurol 12:168-79, (1999).

Cognitive function may also be measured using imaging techniques such as Positron Emission Tomography (PET), functional magnetic resonance imaging (fMRI), Single Photon Emission Computed Tomography (SPECT), or any other imaging technique that allows one to measure brain function. In animals, cognitive function may also be measured with electrophysiological techniques.

“Cognitive decline” or “cognitive impairment” refers to cognitive function in subjects that is not as robust as that expected in an age-matched normal subject (i.e. subjects with mean scores for a given age in a cognitive test) or as that expected in young adult subjects. In some cases, cognitive function is reduced by about 5%, about 10%, about 30%, or more, compared to cognitive function expected in an age-matched normal subject. In some cases, cognitive function is as expected in an age-matched normal subject, but reduced by about 5%, about 10%, about 30%, about 50% or more, compared to cognitive function expected in a young adult subject. Age-related impaired cognitive function may be associated with Mild Cognitive Impairment (MCI), Age-Associated Memory Impairment (AAMI), and Age-related Cognitive Decline (ARCD). Cognitive decline may also be associated with a neurodegenerative disease.

In some embodiments, an individual of the invention may suffer or is at risk of developing memory loss or cognitive decline. Exemplary risk factors include, but are not limited to, genetics factors (e.g., ApoE4) or other risk factors (pre-diabetes, diabetes type 2, hypertension, obesity, etc.).

Memory loss can be tested by neuropsychological testing, on-line tests such as CNS Vital Signs or Lumosity, etc., history from family members or friends; or other cognitive changes such as aphasia, dyscalculia, agnosias, apraxias, spatial memory loss, navigation difficulty, executive function loss; or neuropsychological symptoms such as depression or hyper-irritability, etc.; or at risk determined by genetics (e.g., ApoE4) or other risk factors (pre-diabetes, diabetes type 2, hypertension, obesity, etc.); or imaging (abnormal PET scan suggestive of AD, abnormal MRI with loss of volume of hippocampus or other brain region; or amyloid imaging positive; or retinal scanning showing amyloid; or neural exosomes showing signature of AD.

In some embodiments, an individual of the invention may suffer or is at risk of developing a neurodegenerative disease. Exemplary neurodegenerative diseases include: Alzheimer's disease, Amyotrophic Lateral Sclerosis (ALS), and Parkinson's disease. Another class of neurodegenerative diseases includes diseases caused at least in part by aggregation of poly-glutamine. Diseases of this class include: Huntington's Diseases, Spinalbulbar Muscular Atrophy (SBMA or Kennedy's Disease) Dentatorubropallidoluysian Atrophy (DRPLA), Spinocerebellar Ataxia 1 (SCA1), Spinocerebellar Ataxia 2 (SCA2), Machado-Joseph Disease (MJD; SCA3), Spinocerebellar Ataxia 6 (SCA6), Spinocerebellar Ataxia 7 (SCAT), and Spinocerebellar Ataxia 12 (SCA12).

In some cases, an individual of the invention may suffer or is at risk of developing Alzheimer's disease.

Diagnosis of AD is routine in the art. Doctors generally use a variety of assessments and laboratory measurements to make what we call a “differential diagnosis.” Diagnosing Alzheimer's will likely involve several types of evaluations. Evaluations commonly performed include:

1. Medical history: an interview or questionnaire to identify past medical problems, difficulties in daily activities and any medications (prescriptions, vitamins, supplements and over-the-counter medications), among other things. It is important to inform the doctor of any family history of Alzheimer's or other related medical issues. The doctor may wish to speak to a close family member to supplement information, as it is important to get a thorough picture of a person's medical history.

2. Physical examination: should include evaluations of hearing and sight, heart and lungs, as well as temperature, blood pressure and pulse readings. The doctor might also ask about diet and nutrition and use of alcohol and tobacco products.

3. Standard laboratory tests: might include blood and urine tests designed to help eliminate other possible conditions. These will measure things like blood count, thyroid and liver function, and levels of glucose and other blood-based indicators of illness. A depression screening should also be conducted. In some cases, a small sample of spinal fluid may be collected for testing.

4. Neuropsychological testing: Doctors use a variety of tools to assess memory, problem-solving, attention, vision-motor coordination and abstract thinking, such as performing simple calculations in your head. The goal is to better characterize the types of cognitive symptoms present, which might provide clues to the underlying cause. The most commonly used test is called a mini-mental state exam, or MMSE. During the MMSE, the doctor or health professional will ask a number of questions which test a variety of common mental skills. Some examples of questions on the MMSE will ask about the date or the person's location and also ask the person to count backward or copy a drawn figure.

5. Brain-imaging scan: MRI and CT scans look at the structure of the brain and are used to rule out brain tumors or blood clots in the brain as the reason for symptoms. PET scans can look at how certain parts of the brain are working or how active they are. Many scientists are trying to determine if other brain-imaging techniques might be able to identify telltale signs of early Alzheimer's reliably enough to be used as diagnostic tools.

In some cases, an individual's cognitive function has not been improved on a monotherapy treatment plan. Exemplary monotherapy treatment plan includes, but is not limited to, donepezil and/or memantine and/or rivastigmine and/or galantamine and/or huperzine A and/or a BACE inhibitor and/or an anti-amyloid antibody.

“Treating,” “reducing,” or “reversing” a condition or individual refers to taking steps to obtain beneficial or desired results, including clinical results. Beneficial or desired clinical results include, but are not limited to, alleviation or amelioration of one or more symptoms associated with cognitive impairment/cognitive decline/memory loss, delay or slowing of that impairment, amelioration, palliation or stabilization of that impairment, and other beneficial results, such as improvement of cognitive function or a reduced rate of decline of cognitive function in subjects with cognitive impairment or at risk thereof. Exemplary functional improvements include, but are not limited to, improvement in MoCA (Montreal Cognitive Assessment score) or other neuropsychological testing; improvement in activities of daily living; improvement in any of the symptoms initially reported, such as driving difficulty, math problem, speaking problems, etc.; improvement in the ability to work effectively; improvement in imaging values, such as PET scan or volumetrics of MRI scans; improvement in reduction of episodes of confusion or disorientation; or improvement in any factors listed in Table 1 and Table 2.

Exemplary therapeutic method may include: (1) eliminating all simple carbohydrates, leading to a weight loss of 20 pounds; (2) eliminating gluten and processed food from the diet, and increasing vegetables, fruits, and non-farmed fish; (3) reducing stress by doing yoga; (4) reducing stress with meditation for 20 minutes twice per day; (5) taking melatonin 0.5 mg po qhs; (6) increasing sleep from 4-5 hours per night to 7-8 hours per night; (7) taking methylcobalamin 1 mg each day; (8) taking vitamin D3 2000 IU each day; (9) taking fish oil 2000 mg each day; (10) taking CoQ₁₀ 200 mg each day; (11) optimizing oral hygiene using an electric flosser and electric toothbrush; (12) reinstating HRT (hormone replacement therapy) that had been discontinued following the WHI report in 2002; (13) fasting for a minimum of 12 hours between dinner and breakfast, and for a minimum of three hours between dinner and bedtime; and/or (14) exercising for a minimum of 30 minutes, 4-6 days per week.

Exemplary therapeutic method may include: (1) fasting for a minimum of three hours between dinner and bedtime, and for a minimum of 12 hours between dinner and breakfast; (2) eliminating simple carbohydrates and processed foods from the diet; (3) increasing consumption of vegetables and fruits, and limiting consumption of fish to non-farmed, and meat to occasional grass-fed beef or organic chicken; (4) taking probiotics; (5) taking coconut oil i tsp bid; (6) exercising strenuously, swimming 3-4 times per week, cycling twice per week, and running once per week; (7) taking melatonin 0.5 mg po qhs, and sleeping as close to 8 hours per night as schedule would allow; (8) taking herbs Bacopa monniera 250 mg, Ashwagandha 500 mg, and turmeric 400 mg each day; (9) taking methylcobalamin 1 mg, methyltetrahydrofolate 0.8 mg, and pyridoxine-5-phosphate 50 mg each day; (10) taking citicoline 500 mg po bid; (11) taking vitamin C 1 g per day, vitamin D3 5000 IU per day, vitamin E 400 IU per day, CoQ₁₀ 200 mg per day, Zn picolinate 50 mg per day, and α-lipoic acid 100 mg per day; and/or (12) taking DHA (docosahexaenoic acid) 320 mg and EPA (eicosapentaenoic acid) 180 mg per day.

Exemplary therapeutic method may include: (1) fasting for a minimum of three hours between dinner and bedtime, and for a minimum of 12 hours between dinner and breakfast; (2) eliminating simple carbohydrates and processed foods from the diet; (3) increasing consumption of vegetables and fruits, limiting consumption of fish to non-farmed, and not eating meat; (4) exercising 4-5 times per week; (5) taking melatonin 0.5 mg po qhs, and sleeping as close to 8 hours per night as schedule would allow; (6) reducing stress with meditation and relaxation; (7) taking methylcobalamin 1 mg 4×/wk and pyridoxine-5-phosphate 20 mg each day; (8) taking citicoline 200 mg each day; (9) taking vitamin D3 2000 IU per day and CoQ₁₀ 200 mg per day; (10) taking DHA 700 mg and EPA 500 mg bid; (11) taking prescribed bioidentical estradiol with estriol (BIEST), and progesterone (under health provider's instruction); and/or (12) reducing bupropion from 150 mg per day to 150 mg 3×/wk (under health provider's instruction).

### Systems

In one aspect, a computer-implemented method, a system, and a machine-readable medium comprising computer program instructions, for detecting, managing and/or treating cognitive decline in a patient, is described. One of more data processors forming at least one computing device can be provided for performing the operations described herein. While some of the operations or processes may be described as being performed by a single processor or group of processors, it is contemplated that the operations or processes described herein may be performed by multiple different processors. The multiple different processors may be logically and physically separate, or may be co-located.

In some variations, the operations or processes can include receiving patient parameters of a patient. The patient parameters can be associated with a set of physiological characteristics of the patient. The set of physiological characteristics of the patient can include one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59) of the factors provided in Table 1 and Table 2, above. In some variations, the set of physiological characteristics can include at least 6 of the factors provided in Table 1 and Table 2, above.

In some embodiments, one of the factors of the methods may include insulin resistance, inflammation level, hormone status, homocysteine level, methylation level, metal status, cytoprotection level, or any combination thereof.

In some embodiments, one of the factors of the methods may include hs-CRP level, homocysteine level, vitamin D level, hormone status, albumin:globulin ratio, serum albumin level, glucose status, metal status, alcohol use, history of head trauma, history of drug use, current use of neuroactive medications, ApoE4 status or any combination thereof.

In some embodiments, the insulin resistance factor can include glucose status, fasting blood sugar level or any combination thereof.

In some embodiments, the inflammation level can include cs-CRP level, arachidonic acid (AA)/eicosapentaenoic acid (EPA) ratio, the liver function, meningitis, or any combination thereof. In embodiments, the inflammation level can include cs-CRP level, arachidonic acid (AA)/eicosapentaenoic acid (EPA) ratio or combination thereof.

In some embodiments, the hormone status can include vitamin D3 level, estradiol level, progesterone level, testosterone level, free T3 level, free T4 level, reverse T3 level, TSH level, pregnenolone level, DHEA level, morning cortisol level or any combination thereof.

In some embodiments, the cytoprotection level can include heavy metal toxicity, mitochondrial function, methylation status or any combination thereof.

In some embodiments, the glucose status can include fasting glucose level, fasting insulin level, hemoglobin A1c level or any combination thereof.

In some embodiments, the metal status can include Cu:Zn ratio, RBC Mg level, serum Zn level, RBC Zn level, serum Cu level, heavy metal toxicity or any combination thereof.

The set of physiological parameters can include results of tests performed to measure the physiological characteristics of the patient. The tests performed can include the tests described herein for monitoring or measuring the physiological characteristics of the patient.

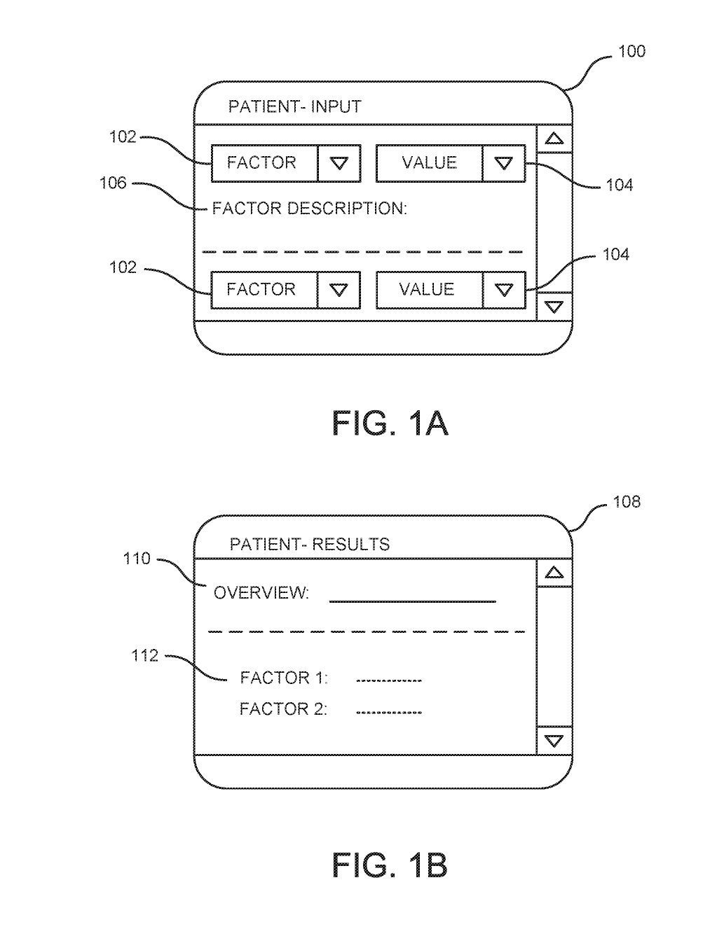

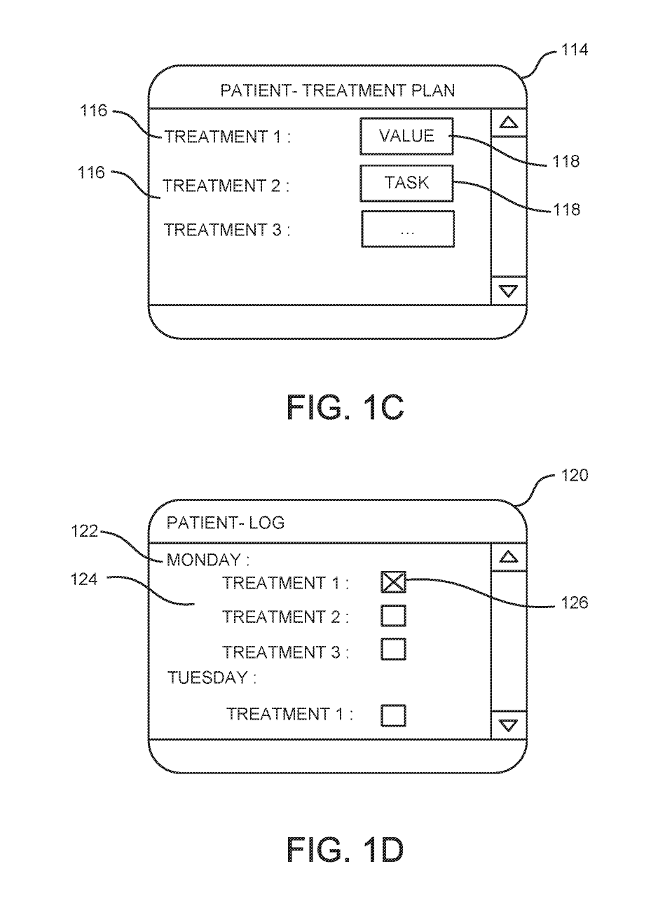

With reference to FIG. 1A, a graphical user interface 100 is illustrated having features consistent with the present description is provided. The graphical user interface 100 may be provided to healthcare professionals to facilitate entry of a patient's physiological characteristics. The graphical user interface 100 can facilitate selection and/or entry of at least one factor 102. The factors 102 that can be selected and/or entered can include the factors described in Table 1 and Table 2, above. The graphical user interface 100 can also facilitate the entry of a value 104. The value 104 can represent the results of a test to determine the state of a patient's physiological characteristics.

In some variations, the graphical user interface 100 can include a factor description 106. The factor description 106 can include information about the factor or physiological characteristic. The factor description 106 can include instructions on the performance of the test to measure or monitor the state of that factor of the patient. The factor description 106 can include information about the relative significance of the factor relative to other factors.

The received patient parameters can be compared against predefined ranges for the set of physiological characteristics. The predefined ranges can include the ranges provided in Table 1 and Table 2

A memory loss risk factor can be determined for the patient based on the comparison. The memory loss risk factor can provide an indication of the current and/or future severity of a patient's cognitive decline. The memory loss risk factor can be based on the number of factors where the patient is outside of the predefined ranges. In some variations, the factors that contribute to the determination of the memory loss risk factor can be weighted. One or more of the factors can be a greater indicator of cognitive decline than other factors. Consequently, when a patient falls outside of acceptable ranges for that factor, the factor contributes to the memory loss risk factor to a greater degree than the other factors.

The determined memory loss risk factor may be a single score. The determined memory loss risk factor may be a combination of scores, presented as an aggregate or individually. The patient parameters that exceed the predefined ranges for the associated physiological characteristics can be aggregated to facilitate provision of the memory loss risk factor. The memory loss risk factor can be a binary indicator of cognitive decline. For example, based on the type and/or number of factors for which the patient exceeds the predefined acceptable range, the patient may be identified as being at-risk, or not at-risk for cognitive decline. In some variations, the memory loss risk factor may provide an indication of a trajectory of cognitive decline.

With reference to FIG. 1B, a graphical user interface 108 is illustrated having features consistent with the present description is provided. The graphical user interface 108 can be configured to provide an indication of an overview 110 of the patient's results. The overview 110 can include an indication of the determined memory loss risk factor. The graphical user interface 108 can include an indication of individual factor results 112. The individual factor results 112 can include an indication of the significance of that particular factor to the overall overview 110 of the patient's results. The individual factor results 112 can include an indication of the amount at which the patient exceeds, or falls within, the acceptable range for that particular factor and provide an indication of how that affects the overall overview 110 of the patient's results.

In some variations, the processes can include determining a memory loss treatment plan based on the patient parameters that exceed the predefined ranges for the associated physiological characteristics. The memory loss treatment plan can be presented through a graphical user interface to a healthcare professional and/or the patient.

With reference to FIG. 1C, a graphical user interface 114 is illustrated having features consistent with the present description is provided. The graphical user interface 114 can facilitate presentation of an indication of the treatment plan for the patient based on the determined memory loss risk factor. The graphical user interface 114 can facilitate presentation of individual treatments 116 for the patient. The individual treatments 116 can include a treatment parameter 118. As an example, the treatment parameter 118 can include an amount of a particular medication, a task to be performed, and/or other treatment parameters associated with the treatment(s) 116.

With reference to FIG. 1D, a graphical user interface 120 is illustrated having features consistent with the present description is provided. The graphical user interface 120 can facilitate maintaining a patient log. The graphical user interface 120 can be presented to the patient for entry by the patient. One or more auxiliary devices can be in electronic communication with a computing device facilitate presentation of the graphical user interface 120. The auxiliary device(s) can be configured to monitor patient compliance with the treatment plan and facilitate provision of an indication of compliance by the patient in the patient log. The patient log can include a record of the patient's adherence to the determined treatment plan. The patient log can include an indication of the day or schedule 122 for the treatment plan. The patient log can include an indication of the treatment(s) 124 to be performed in accordance with the schedule 122. The patient log can include an indication 126 of successful completion of the treatment(s) 124.

In some variation, the graphical user interface 120 presented to the patient can include one or more cognitive tests for completion by the patient. The cognitive tests can be configured to provide an indication of the patient's cognitive abilities. The cognitive tests can be used to facilitate determination the efficacy of the treatment plan.

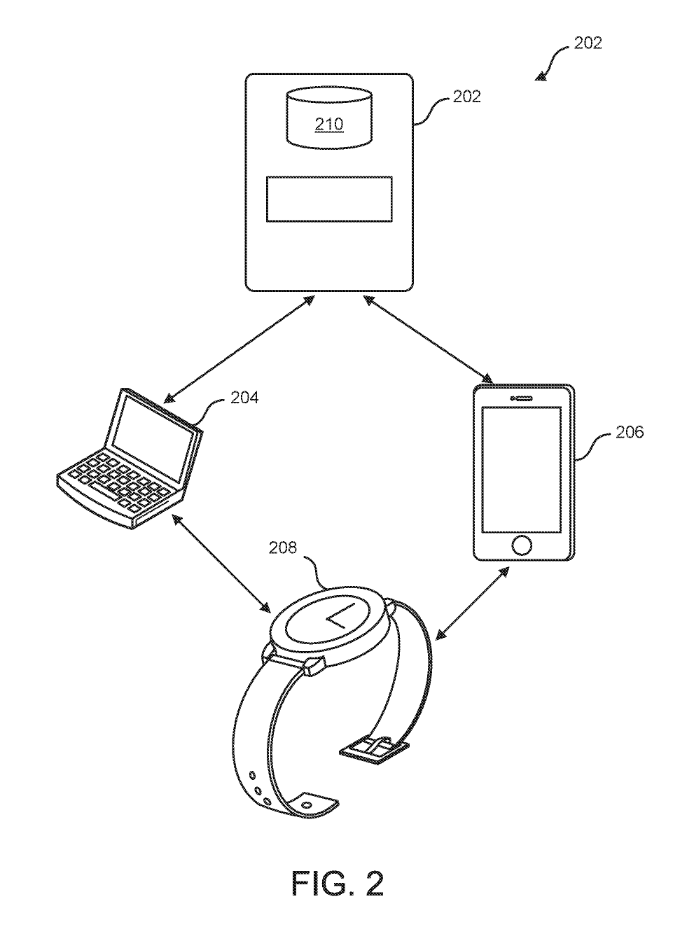

FIG. 2 is a diagram illustrating aspects of a system 200 showing features consistent with implementations of the present description. The system 200 can include one or more server(s) 202. The system 200 can include one or more computing devices 204, 206. The computing devices 204, 206 can be in electronic communication with server(s) 202. In some variations, computing devices 204, 206 can be in electronic communication with server(s) 202 through one or more web servers, and/or other servers and communication systems. While computing devices 204 and 206 are illustrated as being particular computing devices in FIG. 2 the present description contemplates that the computing devices 204 and 206 can be any type of computing device, including a personal computer, a server, a mobile computing device, a wearable computing device, and/or other computing device.

Computing device 204 can include a computing device that used and/or accessed by healthcare professionals. The computing device 204 can be configured to present graphical user interfaces 100, 108, 114 and other graphical user interfaces and the functionality described with reference to graphical user interfaces 100, 108, 114 and other graphical user interfaces.

Computing device 206 can include a computing device that is used and/or accessed by a patient. The computing device 206 can be configured to present graphical user interface 120 and/or other graphical user interfaces to the patient, and provide the functionality described with reference to graphical user interface 120 and/or other graphical user interfaces.

The system 200 can include one or more auxiliary device(s) 208. Auxiliary device(s) 208 can be in electronic communication, intermittently, continuously, or otherwise, with computing device(s) 204 and/or 206. The auxiliary device(s) 208 can be configured to facilitate determination of factors, such as factors described in Table 1 and/or Table 2. The auxiliary device(s) 208 can be configured to facilitate determination of patient compliance with a determined treatment plan. The auxiliary device(s) 208 can be configured to facilitate administration of a determined treatment plan.

The computing device(s) 204 and/or 206 and auxiliary device(s) 208 can include computer-readable instructions that facilitate those computing device(s) to perform one or more of the operations or processes described herein. Over time the operations or processes may evolve. The system 200 may be configured to facilitate update of the computer-readable instructions on the computing device(s) 204 and/or 206 to provide the new functionality attributable to the evolved operations and/or processes. The server(s) 202 can be configured to facilitate provision of the updated computer-readable instructions to the computing device(s) 204 and/or 206 over one or more electronic communication systems.

The server(s) 202 can include electronic storage 210. Electronic storage 210 can be co-located with server(s) 202 or can be physically and/or logically separate from server(s) 202. The server(s) 202 can be configured to receive data from computing device(s) 204 and/or 206. The computing device(s) 204 and/or 206 can be configured to facilitate encryption of the data being transmitted to the server(s) 202. The data received at server(s) can include information associated with the patient's results, treatment plan, adherence to treatment plans, performance on cognitive ability tests, improvements, and/or other information. The received data can be aggregated and used to improve detection and treatment of cognitive decline.

One or more aspects or features of the present description can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs and/or machine-readable instructions, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a touchscreen device, a touchpad a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the claims.

The following examples are provided as illustrations of various embodiments of the invention but are not meant to limit the invention in any manner.

### EXAMPLES

### Example 1 Pilot Study of Using Specific Biological Cognitive Functions in Treating Cognitive Decline

This study demonstrated a novel, comprehensive, and personalized therapeutic program that is based on the underlying pathogenesis of Alzheimer's disease, which is involves multiple modalities designed to achieve metabolic enhancement for neurodegeneration (MEND).

Patients Inclusion Criteria: individuals with memory loss associated with Alzheimer's disease (AD), amnestic mild cognitive impairment (aMCI), or subjective cognitive impairment (SCI), or at risk based on family history or genotype or lifestyle or potential for toxin exposure.

Memory loss was assessed by known methods available in the art, for example, the General Practitioner assessment of Cognition (GPCOG) screening test, memory impairment screen (MIS), the Mini-Cog™, clock drawing test, neuropsychological testing, on-line tests such as CNS Vital Signs or Lumosity, etc., history from family members or friends; or other cognitive changes such as aphasia, dyscalculia, agnosias, apraxias, spatial memory loss, navigation difficulty, executive function loss; or neuropsychological symptoms such as depression or hyper-irritability, etc.; or at risk determined by genetics (e.g., ApoE4) or other risk factors (pre-diabetes, diabetes type 2, hypertension, obesity, etc.); or imaging (abnormal PET scan suggestive of AD, abnormal MRI with loss of volume of hippocampus or other brain region; or amyloid imaging positive; or retinal scanning showing amyloid); or neural exosomes showing signature of AD.

Components of the personalized therapeutic program of this study are summarized in the Table 3 below.

TABLE 3
Components of the therapeutic program

| Components | Approach |
|---|---|
| Optimize diet: minimize | Patients given choice of |
| simple CHO, minimize | several low glycemic, low |
| inflammation, | inflammatory, low grain |
| | diets. |
| Enhance autophagy, | Fast 12 hr each night, |
| ketogenesis | including 3 hr prior to |
| | bedtime. |
| Reduce stress | Personalized-yoga or |
| | meditation or music, etc. |
| Optimize sleep | 8 hr sleep per night; |
| | melatonin 0.5 mg po qhs; Trp |
| | 500 mg po 3x/wk if |
| | awakening. Exclude sleep |
| | apnea. |
| Exercise | 30-60′ per day, 4-6 days/wk |
| Brain stimulation | Posit or related |
| Homocysteine <7 | Me-B12, MTHF, P5P; TMG |
| | if necessary |
| Serum B12 >500 | Me-B12 |
| CRP <1.0; A/G >1.5 | Anti-inflammatory diet; |
| | curcumin; DHA/EPA; |
| | optimize hygiene |
| Fasting insulin <7; HgbA1c | Diet as above |
| <5.5 | |
| Hormone balance | Optimize fT3, fT4, E2, T, |
| | progesterone, pregnenolone, |
| | cortisol |
| GI health | Repair if needed; prebiotics |
| | and probiotics |
| Reduction of A-beta | Curcumin, Ashwagandha |
| Cognitive enhancement | Bacopa monniera, MgT |
| 250H-D3 = 50-100 ng/ml | Vitamins D3, K2 |
| Increase NGF | H. erinaceus or ALCAR |
| Provide synaptic structural | Citicoline, DHA |
| components | |
| Optimize antioxidants | Mixed tocopherols and |
| | tocotrienols, Se, blueberries, |
| | NAC, ascorbate, α-lipoic acid |
| Optimize Zn:fCu ratio | Depends on values obtained |
| Ensure nocturnal | Exclude or treat sleep apnea |
| oxygenation | |
| Optimize mitochondrial | CoQ or ubiquinol, α-lipoic |
| function | acid, PQQ, NAC, ALCAR, |
| | Se, Zn, resveratrol, ascorbate, |
| | thiamine |
| Increase focus | Pantothenic acid |
| Increase SirT1 function | Resveratrol |
| Exclude heavy metal toxicity | Evaluate Hg, Pb, Cd; chelate |
| | if indicated |
| MCT effects | Coconut oil or Axona |

CHO, carbohydrates;
Hg, mercury;
Pb, lead;
Cd, cadmium;
MCT, medium chain triglycerides;
PQQ, polyquinoline quinone;
NAC, N acetyl cysteine;
CoQ, coenzyme Q;
ALCAR, acetyl L carnitine;
DHA, docosahexaenoic acid;
MgT, magnesium threonate;
fT3, free triiodothyronine;
fT4, free thyroxine;
E2, estradiol;
T, testosterone;
Me B12, methylcobalamin;
MTHF, methyltetrahydrofolate;
P5P, pyridoxal 5 phosphate;
TMG, trimethylglycine;
Trp, tryptophan

Exemplary Personalized Therapeutic Programs

1. Subject One

Patient one is a 67-year-old woman presented with two years of progressive memory loss. She held a demanding job that involved preparing analytical reports and traveling widely, but found herself no longer able to analyze data or prepare the reports, and therefore was forced to consider quitting her job. She noted that when she would read, by the time she reached the bottom of a page she would have to start at the top once again, since she was unable to remember the material she had just read. She was no longer able to remember numbers, and had to write down even 4-digit numbers to remember them. She also began to have trouble navigating on the road: even on familiar roads, she would become lost trying to figure out where to enter or exit the road. She also noticed that she would mix up the names of her pets, and forget where the light switches were in her home of years.

When the patient consulted her physician about her problems, she was told that she had the same problem her mother had had, and that there was nothing he could do about it. Her mother had developed similar progressive cognitive decline beginning in her early 60s, had become severely demented, entered a nursing home, and died at approximately 80 years of age.

She began the program, and was able to adhere to some but not all of the protocol components. Nonetheless, after three months she noted that all of her symptoms had abated: she was able to navigate without problems, remember telephone numbers without difficulty, prepare reports and do all of her work without difficulty, read and retain information, and, overall, she became asymptomatic. She noted that her memory was now better than it had been in many years. On one occasion, she developed an acute viral illness, discontinued the program, and noticed a decline, which reversed when she reinstated the program. Two and one-half years later, now age 70, she remains asymptomatic and continues to work full-time.

The components of the program that this patient followed are: (1) she eliminated all simple carbohydrates, leading to a weight loss of 20 pounds; (2) she eliminated gluten and processed food from her diet, and increased vegetables, fruits, and non-farmed fish; (3) in order to reduce stress, she began yoga, and ultimately became a yoga instructor; (4) as a second measure to reduce the stress of her job, she began to meditate for 20 minutes twice per day; (5) she took melatonin 0.5 mg po qhs; (6) she increased her sleep from 4-5 hours per night to 7-8 hours per night; (7) she took methylcobalamin 1 mg each day; (8) she took vitamin D3 2000 IU each day; (9) she took fish oil 2000 mg each day; (10) she took CoQ₁₀ 200 mg each day; (11) she optimized her oral hygiene using an electric flosser and electric toothbrush; (12) following discussion with her primary care provider, she reinstated HRT (hormone replacement therapy) that had been discontinued following the WHI report in 2002; (13) she fasted for a minimum of 12 hours between dinner and breakfast, and for a minimum of three hours between dinner and bedtime; (14) she exercised for a minimum of 30 minutes, 4-6 days per week.

2. Patient Two

Patient two is a 69-year-old entrepreneur and professional man presented with 11 years of slowly progressive memory loss, which had accelerated over the past one or two years. In 2002, at the age of 58, he had been unable to recall the combination of the lock on his locker, and he felt that this was out of the ordinary for him. In 2003, he had FDG-PET (fluoro-deoxyglucose positron emission tomography), which was read as showing a pattern typical for early Alzheimer's disease, with reduced glucose utilization in the parietotemporal cortices bilaterally and left>right temporal lobes, but preserved utilization in the frontal lobes, occipital cortices, and basal ganglia. In 2003, 2007, and 2013, he had quantitative neuropsychological testing, which showed a reduction in CVLT (California Verbal Learning Test) from 84% ile to 1% ile, a Stroop color test at 16% ile, and auditory delayed memory at 13% ile. In 2013, he was found to be heterozygous for ApoE4 (3/4). He noted that he had progressive difficulty recognizing the faces at work (prosopagnosia), and had to have his assistants prompt him with the daily schedule. He also recalled an event during which he was several chapters into a book before he finally realized that it was a book he had read previously. In addition, he lost an ability he had had for most of his life: the ability to add columns of numbers rapidly in his head.

He had a homocysteine of 18 μmol/l, CRP<0.5 mg/l, 25-OH cholecalciferol 28 ng/ml, hemoglobin A1c 5.4%, serum zinc 78 mcg/dl, serum copper 120 mcg/dl, ceruloplasmin 25 mg/dl, pregnenolone 6 ng/dl, testosterone 610 ng/dl, albumin:globulin ratio of 1.3, cholesterol 165 mg/dl (on Lipitor), HDL 92, LDL 64, triglyceride 47, AM cortisol 14 mcg/dl, free T3 3.02 pg/ml, free T4 1.27 ng/l, TSH 0.58 mIU/l, and BMI 24.9.

He began on the therapeutic program, and after six months, his wife, co-workers, and he all noted improvement. He lost 10 pounds. He was able to recognize faces at work unlike before, was able to remember his daily schedule, and was able to function at work without difficulty. He was also noted to be quicker with his responses. His life-long ability to add columns of numbers rapidly in his head, which he had lost during his progressive cognitive decline, returned. His wife pointed out that, although he had clearly shown improvement, the more striking effect was that he had been accelerating in his decline over the prior year or two, and this had been completely halted.

This patient began on the following components of the program: (1) he fasted for a minimum of three hours between dinner and bedtime, and for a minimum of 12 hours between dinner and breakfast; (2) he eliminated simple carbohydrates and processed foods from his diet; (3) he increased consumption of vegetables and fruits, and limited consumption of fish to non-farmed, and meat to occasional grass-fed beef or organic chicken; (4) he took probiotics; (5) he took coconut oil i tsp bid; (6) he exercised strenuously, swimming 3-4 times per week, cycling twice per week, and running once per week; (7) he took melatonin 0.5 mg po qhs, and tried to sleep as close to 8 hours per night as his schedule would allow; (8) he took herbs Bacopa monniera 250 mg, Ashwagandha 500 mg, and turmeric 400 mg each day; (9) he took methylcobalamin 1 mg, methyltetrahydrofolate 0.8 mg, and pyridoxine-5-phosphate 50 mg each day; (10) he took citicoline 500 mg po bid; (11) he took vitamin C 1 g per day, vitamin D3 5000 IU per day, vitamin E 400 IU per day, CoQ₁₀ 200 mg per day, Zn picolinate 50 mg per day, and α-lipoic acid 100 mg per day; (12) he took DHA (docosahexaenoic acid) 320 mg and EPA (eicosapentaenoic acid) 180 mg per day.

3. Patient Three

Patient three is a 55-year-old attorney suffered progressively severe memory loss for four years. She accidentally left the stove on when she left her home on multiple occasions, and then returned, horrified to see that she had left it on once again. She would forget meetings, and agree to multiple meetings at the same time. Because of an inability to remember anything after a delay, she would record conversations, and she carried an iPad on which she took copious notes (but then forgot the password to unlock her iPad). She had been trying to learn Spanish as part of her job, but was unable to remember virtually anything new. She was unable to perform her job, and she sat her children down to explain to them that they could no longer take advantage of her poor memory, that instead they must understand that her memory loss was a serious problem. Her children noted that she frequently became lost in mid-sentence, that she was slow with responses, and that she frequently asked if they had followed up on something she thought she had asked them to do, when in fact she had never asked them to do the tasks to which she referred.

Her homocysteine was 9.8 μmol/l, CRP 0.16 mg/l, 25-OH cholecalciferol 46 ng/ml, hemoglobin A1c 5.3%, pregnenolone 84 ng/dl, DHEA 169 ng/dl, estradiol 275 pg/ml, progesterone 0.4 ng/ml, insulin 2.7 μIU/ml, AM cortisol 16.3 mcg/dl, free T3 3.02 pg/ml, free T4 1.32 ng/l, and TSH 2.04 mIU/I

After five months on the therapeutic program, she noted that she no longer needed her iPad for notes, and no longer needed to record conversations. She was able to work once again, was able to learn Spanish, and began to learn a new legal specialty. Her children noted that she no longer became lost in mid-sentence, no longer thought she had asked them to do something that she had not asked, and answered their questions with normal rapidity and memory.

The treatment program she is following includes the following components: (1) she fasted for a minimum of three hours between dinner and bedtime, and for a minimum of 12 hours between dinner and breakfast; (2) she eliminated simple carbohydrates and processed foods from her diet; (3) she increased consumption of vegetables and fruits, limited consumption of fish to non-farmed, and did not eat meat; (4) she exercised 4-5 times per week; (5) she took melatonin 0.5 mg po qhs, and tried to sleep as close to 8 hours per night as her schedule would allow; (6) she tried to reduce stress in her life with meditation and relaxation; (7) she took methylcobalamin 1 mg 4×/wk and pyridoxine-5-phosphate 20 mg each day; (8) she took citicoline 200 mg each day; (9) she took vitamin D3 2000 IU per day and CoQ₁₀ 200 mg per day; (10) she took DHA 700 mg and EPA 500 mg bid; (11) her primary care provider prescribed bioidentical estradiol with estriol (BIEST), and progesterone; (12) her primary care provider worked with her to reduce her bupropion from 150 mg per day to 150 mg 3×/wk.

Overall Outcome of the Study:

TABLE 4
Summary of patients treated with the therapeutic system described

| Patient | History, evaluation | Diagnosis | Status |
|---|---|---|---|
| 67 F 3/3 | 2 yr memory ↓; FH+ | aMCI | Normal x 2.5 yrs; |
| | | | working |
| 69 M 4/3 | 12 yr memory ↓; | Early AD | “Clearly improved;” |
| | FDG-PET+, | | working |
| | NPsych+ | | |
| 70 M 4/3 | 4 yr memory ↓; | AD | Improved; MemTrax |
| | NPsych+, failed | | passed |
| | MemTrax | | |
| 75 M 3/3 | 1 yr memory ↓ | SCI | Improved; working |
| 75 F C677T | 1 yr memory ↓ | aMCI/early AD | Improved |
| 55 F 3/3 | 4 yr memory ↓ | aMCI/early AD | Normal; working |
| 72 M 3/3 | 7 yr memory ↓ | aMCI | Improved; working |
| 55 M 4/3 | 2 yr memory ↓ | SCI | Normal; working |
| 63 F 4/3 | FH dementia, mild | SCI | Normal, negative |
| | memory ↓, | | amyloid PET; |
| | | | working |
| 60 F 4/3 | 4 yr rapid decline; | Late AD | Decline |
| | MoCA 6, amyloid | | |
| | PET+ | | |

F, female;
M, male;
3/3, ApoE 3/3;
4/3, ApoE 4/3;
C677T, the C677T mutation in methylene tetrahydrofolate reductase (MTHFR);
FH, family history;
aMCI, amnestic mild cognitive impairment;
SCI, subjective cognitive impairment;
FDG PET+ fluorodeoxyglucose positron emission tomography interpreted as typical of Alzheimer's disease;
amyloid PET+, amyloid PET scan read as abnormal, indicative of amyloid accumulation;
NPsych+, quantitative neuropsychology tests showing abnormalities typical of AD;
MoCA, Montreal Cognitive Assessment;
MemTrax, an iPhone application that quantitates memory.

Nine of the 10 displayed subjective or objective improvement in cognition beginning within 3-6 months, with the one failure being a patient with very late stage AD. Six of the patients had had to discontinue working or were struggling with their jobs at the time of presentation, and all were able to return to work or continue working with improved performance. Improvements have been sustained, and at this time the longest patient follow-up is two and one-half years from initial treatment, with sustained and marked improvement.

Results from this study have demonstrated that memory loss in patients with subjective cognitive impairment, mild cognitive impairment, and at least the early phase of Alzheimer's disease, may be reversed, and improvement sustained, with the therapeutic program described here. This is the first such demonstration.

1. A method of treating, reducing or reversing cognitive decline in an individual comprising (a) assessing at least 6 factors of Table 1; (b) optimizing these factors if they are abnormal, wherein the optimizing is achieved by performing one or more optimization approaches associated with the factors, wherein said factors comprise insulin resistance, inflammation level, hormone status, homocysteine level, methylation status, metal status, cytoprotection level or any combination thereof.

2. A method of treating, reducing or reversing cognitive decline in an individual comprising (a) assessing at least 6 factors of Table 1; (b) optimizing these factors if they are abnormal, wherein the optimizing is achieved by performing one or more optimization approaches associated with the factors.

3. The method of claim 1, wherein said method comprising assessing at least 7 factors of Table 1.

4. The method of claim 1, wherein said method comprising assessing at least 8 factors of Table 1.

5. The method of claim 1, wherein said method comprising assessing at least 9 factors of Table 1.

6. The method of claim 1, wherein said method comprising assessing at least 10 factors of Table 1.

7. The method of claim 1, wherein said factors comprise insulin resistance, inflammation level, hormone status, homocysteine level, methylation status, metal status, cytoprotection level or any combination thereof.

8. The method of claim 6, wherein the at least 10 factors comprises hs-CRP level, homocysteine level, vitamin D level, hormone status, albumin:globulin ratio, serum albumin level, glucose status, metal status, alcohol use, history of head trauma, history of drug use, current use of neuroactive medications, ApoE4 status or any combination thereof.

9. The method of claim 1, wherein the insulin resistance factor comprises glucose status, fasting blood sugar level or any combination thereof.

10. The method of claim 1, wherein the inflammation level comprises cs-CRP level, arachidonic acid (AA)/eicosapentaenoic acid (EPA) ratio, meningitis or any combination thereof.

11. The method of claim 1, wherein the hormone status comprises estradiol level, progesterone level, testosterone level, free T3 level, free T4 level, reverse T3 level, TSH level, pregnenolone level, DHEA level, morning cortisol level, vitamin D3 level or any combination thereof.

12. The method of claim 1, wherein the cytoprotection level comprises heavy metal toxicity, mitochondrial function, methylation status or any combination thereof.

13. The method of claim 1, wherein the glucose status comprises fasting glucose level, fasting insulin level, hemoglobin A1c level or any combination thereof.

14. The method of claim 1, wherein the metal status comprises Cu:Zn ratio, RBC Mg level, serum Zn level, RBC Zn level, serum Cu level, heavy metal toxicity or any combination thereof.

15. The method of claim 1, wherein the individual has memory loss.

16. The method of claim 1, wherein the individual has a family history of neurodegenerative disease.

17. The method of claim 1, wherein the individual has a neurodegenerative disease.

18. The method of claim 1, wherein the individual has Alzheimer's disease.

19. The method of claim 1, wherein the individual's health has not been improved on a monotherapy treatment plan.

20. The method of claim 19, wherein the monotherapy treatment plan comprises donepezil, memantine, rivastigmine, galantamine, huperzine A, a BACE inhibitor, an anti-amyloid antibody or any combination thereof.

21. A method of identifying additional treatment modalities in individuals in need thereof comprising (a) assessing at least 6 factors of Table 1; (b) identifying the factors that are abnormal; and (c) providing additional treatment modality to match the factor(s) of step (b).

22. A method for metabolic enhancement of neurodegeneration in an individual comprising (a) assessing at least 6 factors of Table 1; (b) optimizing these factors if they are abnormal, wherein the optimizing is achieved by performing one or more optimization approaches associated with the factors.

23. The method of claim 21, wherein said method comprising assessing at least 7 factors of Table 1.

24. The method of claim 21, wherein said method comprising assessing at least 8 factors of Table 1.

25. The method of claim 21, wherein said method comprising assessing at least 9 factors of Table 1.

26. The method of claim 21, wherein said method comprising assessing at least 10 factors of Table 1.

27. The method of claim 21, wherein said factors comprises insulin resistance, inflammation level, hormone status, homocysteine level, methylation status, metal status, cytoprotection level or any combination thereof.

28. The method of claim 26, wherein the at least 10 factors comprises hs-CRP level, homocysteine level, vitamin D level, hormone status, albumin:globulin ratio, serum albumin level, glucose status, metal status, alcohol use, history of head trauma, history of drug use, current use of neuroactive medications, ApoE4 status or any combination thereof.

29. A computer-implemented method for implementation by one or more data processors forming part of at least one computing device to facilitate treating, reducing or reversing cognitive decline, the method comprising:
receiving, at the one or more data processors, patient parameters of a patient, the patient parameters associated a set of physiological characteristics of the patient;
comparing, at the one or more data processors, the patient parameters with predefined ranges for the set of physiological characteristics; and,
determining, at the one or more data processors, a memory loss risk factor for the patient based on the comparison.

30. The computer-implemented method as in claim 29, wherein the set of physiological characteristics of the patient including at least 6 factors of Table 1.

31. The computer-implemented method of claim 29, further comprising:
determining, by the one or more data processors, a memory loss treatment plan based on the patient parameters that exceed the predefined ranges for the associated physiological characteristics.

32. The computer-implemented method of claim 31, wherein determining a memory loss treatment plan comprises optimizing the set of physiological characteristics if the patient parameters are abnormal, where the optimization is achieved by performing one or more optimization approaches associated with the set of physiological characteristics.

33. The computer-implemented method of claim 31, further comprising:
presenting, through a graphical user interface, the memory loss treatment plan.

34. The computer-implemented method of claim 29, wherein determining the memory loss risk factor further comprises:
determining, for individual ones of the patient parameters an amount that the individual patient parameter exceeds the predefined range for the associated physiological characteristic.

35. The computer-implemented method of claim 29, wherein determining the memory loss risk factor further comprises:
aggregating the patient parameters that exceed the predefined ranges for the associated physiological characteristics.
